(19) United States
(12) Patent Application Publication
(10) Pub. No.: US 2025/0305021 A1
(43) Pub. Date: Oct. 2, 2025

(54) ENZYMATIC LYSIS FOR EXTRACTION OF BIOPRODUCTS FROM YEAST

(71) Applicant: C16 Biosciences, Inc., New York, NY (US)

(72) Inventors: Harold M. MCNAMARA, New York, NY (US); Corentin MOEVUS, New York, NY (US); Jeffrey Li, New York, NY (US); Alexandre CHAPEAUX, New York, NY (US)

(21) Appl. No.: 18/864,597

(22) PCT Filed: May 9, 2023
(86) PCT No.: PCT/US2023/021549

§ 371 Date: Nov. 11, 2024

Related U.S. Application Data
(60) Provisional application No. 63/340,813, filed on May 11, 2022.

(51) Int. Cl. C12P 23/00; C12N 1/06; C12N 1/16; C12N 9/24; C12N 9/42; C12N 9/50; C12R 1/645

(52) U.S. Cl. CPC C12P 23/00; C12N 1/063; C12N 1/165; C12N 9/2437; C12N 9/2494; C12N 9/50; C12R 2001/645; C12Y 302/01004; C12Y 302/01039; C12Y 302/01078

(57) ABSTRACT

The disclosure relates to novel methods, compositions, and genetically modified microorganisms for extracting and/or isolating bioproducts from microorganisms having recalcitrant cell walls. In some aspects, the disclosure relates to solvent-free methods of extracting and/or isolating bioproducts. The disclosure further relates to bioproducts having less than 10 ppm of a solvent.

### CROSS-REFERENCE TO RELATED APPLICATIONS

This application claims the benefit of U.S. Provisional Application No. 63/340,813 filed on May 11, 2022, which is hereby incorporated by reference in its entirety for all purposes.

### FIELD OF THE DISCLOSURE

The present disclosure relates to environmentally friendly and sustainable alternatives to acid-based extraction methods of bioproducts from yeast. The disclosure further relates to solvent free methods of extraction of bioproducts from yeast.

### BACKGROUND

Extracting valuable bioproducts from industrious yeasts can be challenging and expensive. For example, it's been reported that upwards of 70% of the cost of making biodiesel in industrious yeasts is accrued after biomass production, during the extraction step. This is due primarily to recalcitrant cell walls, composed of polymers and assembled for the sole purpose of resisting breakdown. Thus, lysing such a cell to recover these bioproducts can be costly and time consuming. Conventional lysis via acid treatment and solvent recovery is costly, generates waste streams (e.g. acid neutralization byproducts), may require specialized equipment for handling of organic solvents, and can perturb product qualities (e.g. oxidation of the oil during lysis; residual solvent contamination).

Some of the most valuable yeast, such as the red yeast of the Rhodotorula, Rhodosporidium, or Sporobolomyces genera have recalcitrant cells walls; R. toruloides in particular poses a challenge due to the large composition of β-1,3-glucomannose. R. glutinis is reported to have a 4-layer structure composed of 55% glucomannan and 20% fucogalactomannan, with unusual β-(1→4) and β-(1→3) linkages for mannopyranose units (Lee, T. H., et al., Localization of glucomannan and fucogalactomannan in Rhodotorula glutinis cell wall and spheroplast formation of its living cell, 1981, Agric. Biol. Chem., 45(10): 2343-2345).

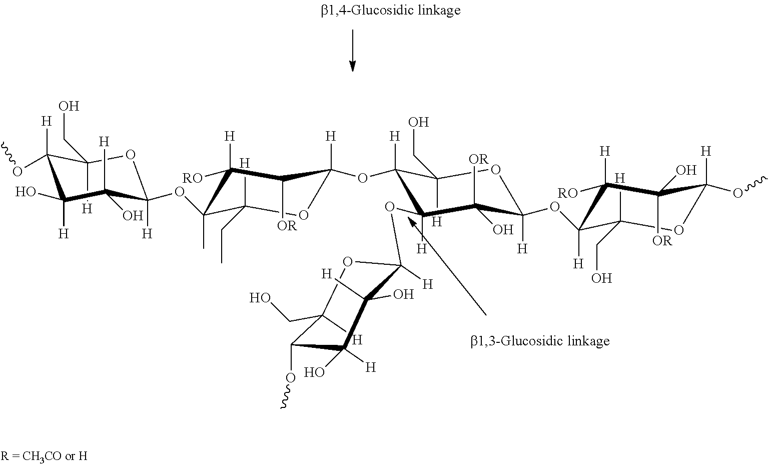

### Structure of 1,3 B-Glucans, 1,6 B-Glucans, and Glucosidic Linkages

Commercial enzymatic lysis reagents (e.g. zymolyase) have little effect on R. toruloides. Other cellulolytic fungi, such as Trichoderma reesei, can completely lyse other oleaginous yeast (Cutaneotrichosporon oleaginosus) but it has been reported to be unable to effectively lyse R. toruloides (Masri, Mahmoud A., et al. “A sustainable, high-performance process for the economic production of waste-free microbial oils that can replace plant-based equivalents.” Energy & Environmental Science 12.9 (2019): 2717-2732).

Some reports have shown that the enzyme plMAN5c (isolated from the fungus P. lilacinum) has activity against R. toruloides (Jin, Guojie, et al. “Enzyme-assisted extraction of lipids directly from the culture of the oleaginous yeast Rhodosporidium toruloides.” Bioresource technology 111 (2012): 378-382; Yang F. et al., “Purification and characterization of a β-1,3-glucomannanase expressed in Pichia pastoris” Enz. And Microb. Technology 2011, 49(2):223-228). This type of enzymatic lysis has the potential to address both the problem of oxidation of the bioproduct (e.g. oil) during lysis, and residual solvent contamination if it is sufficiently complete to enable separation of the bioproduct without the use of solvents. Additionally, the biochemical activity of enzymatic lysis is more targeted than acid lysis, specifically degrading cell wall bonds and leaving the bioproduct unperturbed. However, in order to realize these advantages, it has to remain cost-effective. A process involving recombinant expression and purification of a separate enzyme carries a separate cost burden, and must be efficient to provide a net benefit. Jin, Guojie, et al. 2012 used high concentrations of enzyme (ranging from 0.75 to 3.5 g/kg) with ethyl acetate to achieve lipid extraction, and reported poor efficiency (less than 10%) using hexane as a solvent. Consequently, the current methods of extraction rely on acids or high levels of enzymes plus additional treatments (such as heating or microwave irradiation) and are not cost-effective or environmentally friendly.

Thus, there is an urgent need for alternative methods to recover valuable bioproducts from industrious yeast that is efficient, cost-effective, and leaves the bioproduct unperturbed and free of contamination.

### BRIEF SUMMARY

In one embodiment, the present disclosure teaches methods for isolating a bioproduct from a yeast comprising treating yeast cells with a β-1,3-glucomannanase, wherein the β-1,3-glucomannanase is in an amount of less than 1.0e-4 g enzyme protein/g dry cell weight, thereby producing an enzymatically lysed sample, separating the lipid phase of the enzymatically lysed sample via solvent or non-solvent extraction, thereby producing a separated sample, and isolating a bioproduct from the separated sample.

In another embodiment, the present disclosure teaches methods for enzymatic lysis of microorganisms having recalcitrant cell walls comprising inactivating a biomass of microorganisms having recalcitrant cell walls, inoculating the inactive biomass with live cellulolytic fungi and/or an organism engineered to express at least one cellulolytic enzyme, and incubating the live cellulolytic fungi and/or an organism engineered to express at least one cellulolytic enzyme for at least 5 hours to generate a lysed biomass.

In another embodiment, the present disclosure provides for compositions comprising two or more enzymes, wherein the two or more enzymes comprise an isolated and purified β-1,3-glucomannanase and at least one of a cellulase and a protease, and an inactive cell biomass, wherein the composition has a total grams enzyme to dry cell weight ratio between 1:10,000 and 1:1,000,000.

In another embodiment, the present disclosure provides for compositions comprising a live cellulolytic fungus and an inactive yeast, wherein the cellulolytic fungus is a species selected from Trichoderma, Humicola, Penicillium, Purpureocillium, Phanerochaete, and Pycnoporus, and wherein the inactive yeast is a species selected from Rhodotorula, Rhodosporidium, or Sporobolomyces, and wherein the inactive yeast to live cellulolytic fungus ratio is between 1000:1 and 1:1 dry cell w/w.

In another embodiment, the present disclosure relates to bioproducts produced from the methods disclosed herein and/or isolated from the compositions disclosed herein. In some aspects, the bioproduct does not comprise a detectable amount of a solvent.

In another embodiment, the present disclosure relates to a microbial oil produced by an oleaginous yeast, wherein the oil comprises less than 10 ppm of a solvent, and at least one pigment selected from the group consisting of carotene, torulene and torulorhodin.

In another embodiment, the present disclosure relates to an autolytic yeast that produces a bioproduct, wherein the yeast comprises a gene encoding a cellulolytic enzyme, and wherein expression of the gene is under the control of an inducible promoter.

In another embodiment, the present disclosure teaches autolytic methods for producing a bioproduct from an industrious yeast comprising genetically engineering an industrious yeast to express and/or secrete a cellulolytic enzyme, wherein the industrious yeast produces a bioproduct, and wherein the expression of the cellulolytic enzyme is under the control of an inducible promoter, growing the yeast to produce the bioproduct, inducing expression of the cellulolytic enzyme to autolyse the yeast, and extracting, isolating, and/or purifying the bioproduct.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is a line graph of the amount of the recovered oil titer (g/L) over time (in hours) at 50° C. for different enzyme concentrations.

FIG. 2 shows photographs of a T. reesei (ATCC 56765) halo assay on YPD-based EM agar taken over 6 days. From left to right, the first column shows control plates grown on malt extract agar (MEA). The second through fourth columns show the plates grown on YPD-based EM agar at day 0, 5, and 6, respectively. The YPD-based EM agar contained 50 g/L wet cell weight R. toruloides biomass obtained from culture in rich medium. The plates show clear growth, but no obvious solubilization of the R. toruloides biomass.

FIGS. 3A-3C shows photographs of halo assays for P. lilacinum (ATCC 36010). In FIG. 3A, the plates show lytic activity in both EM agar formulations and growth comparable to that on a rich medium. FIG. 3B shows a timecourse analysis of the halo formation by P. lilacinum on YPD-based EM agar, containing 50 g/L wet cell weight R. toruloides biomass obtained from culture in rich medium. The clear zone expands with the colony perimeter, showing clear solubilization of the R. toruloides biomass over time. YPD overlay and longer incubation resulted in a more visible halo (FIG. 3C).

FIGS. 4A-4C show photographs of P. lilacinum cultures in EM, SEM, and autoclaved DASGIP suspension. FIG. 4A shows the change in appearance after shake-flask culture (day 0), and FIG. 4B shows the change in appearance at day 7. FIG. 4C shows the centrifuge settling patterns; the yellow arrow is pointing to phase separation of free oil.

FIGS. 5A-5C show a comparison of DASGIP suspension with and without live P. lilacinum treatment. FIG. 5A shows side-by-side comparison of samples pelleted at 3500×g. At higher speed (14,000×g) the untreated sample displays the banding from a probable lipid body fraction, but no free oil (FIG. 5B). Pure oil could be obtained via solvent-free extraction, using only gravimetric separation of P. lilacinum treated DASGIP suspension (FIG. 5C).

FIGS. 6A-6B show compositional analyses of oils from P. lilacinum extractions. FIG. 6A is a stacked bar graph of the FAME profiles of R. toruloides oil prepared by HCl-chloroform-methanol method vs by P. lilacinum direct extraction, or P. lilacinum chloroform-methanol extraction. P. lilacinum treatment increased the C18:2 content and decreased the C16:0 content. FIG. 6B shows a thin layer chromatography of TAG content of live-P. lilacinum extracted oil as well as for oil samples from cell mass treated with P. lilacinum EM or P. lilacinum SEM broth, followed by solvent extraction.

FIGS. 7A-7B shows the carotenoid analyses of live P. lilacinum extracted oil (LC-DAD chromatogram). The lack of torularhodin may be explained by the requirement for acidic conditions for solvent extraction due to its pKa (FIG. 7B).

FIGS. 8A-8B show the effects of inoculum choice (FIG. 8A) and physical pretreatment (FIG. 8B) on successful oil phase formation. PL—P. lilacinum, TR—T. reesei.

FIG. 9 is a line graph of the recovered lipids (g/L, y-axis) over time (days, x-axis) of autoclaved R. toruloides biomass incubated with, and without, P. lilacinum.

FIGS. 10A-10B shows stacked bar graphs of fatty acid (FA) (FIG. 10A) and unsaturated (FIG. 10B) profiles of the recovered lipids over time, and compared to the previous (earlier batch) data shown in FIG. 9 (compare stacked bars captured by rectangles in FIG. 10A). Little variance (˜3%) was observed.

FIG. 11 shows photographs of bottles containing extracts from different lytic treatments. Pigment is more pronounced in HCl and PL SEM treated extracts.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

“Fatty acid profile” as used herein refers to how specific fatty acids contribute to the chemical composition of an oil.

“Microorganism” and “microbe” mean any microscopic unicellular organism and can include bacteria, algae, yeast, or fungi.

“Oleaginous” as used herein refers to material, e.g., a microorganism, which contains a significant component of oils, or which is itself substantially composed of oil. An oleaginous microorganism can be one that is naturally occurring or synthetically engineered to generate a significant proportion of oil.

“Industrious yeast” or “industrial yeast” as used herein refers to a collection of yeast species that can accumulate valuable bioproducts.

“Tailored fatty acid profile” as used herein refers to a fatty acid profile in a microbial oil which has been manipulated towards target properties, either by changing culture conditions, the species of oleaginous microorganism producing the microbial oil, or by genetically modifying the oleaginous microorganism.

“W/W” or “w/w”, in reference to proportions by weight, refers to the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

“Recalcitrance” as used herein refers to the intrinsic resistance to breakdown imposed by polymer assembly of the cell wall.

“Bioproduct” as used herein refers to any product produced from or derived from a renewable biological resource.

“Detectable amount of a solvent” as used herein is anything above 0.1 ppm. Thus, an “undetectable” amount would be less than or equal to 0.1 ppm.

“Inactive yeast” as used herein refers to yeast cells that are no longer alive.

“Cellulolytic fungi” or fungus, are fungi capable of breaking down cellulose-containing material.

### Overview

The present disclosure relates to novel methods, compositions, and genetically modified microorganisms for extracting and/or isolating bioproducts from microorganisms having recalcitrant cell walls. The disclosure further relates to bioproducts having less than 10 ppm of a solvent.

### Industrious Yeasts

For thousands of years, yeasts have been put to work to make various fermented foods and beverages. In recent decades, yeasts have been employed for a variety of biotechnical applications. By exploiting their natural diversity, directing evolution, and/or targeting specific metabolic pathways with genetic modifications, industrious yeast lines can produce a wide variety of valuable bioproducts. One such category of industrious yeasts that can be used with the methods and compositions disclosed herein are the “red yeasts” of the Rhodotorula, Rhodosporidium, and Sporobolomyces genera, so named for their distinctive orange/red colored colonies when grown on agar.

### Rhodotorula and Rhodosporidium

The Rhodotorula genus comprises both single cell yeast that reproduce asexually—the Rhodotorula species, as well as species that reproduce sexually and alternate between yeast and filamentous phases—the Rhodosporidium species. This group of industrious yeasts give rise to biofuels, carotenoids, enzymes, biosurfactants, and can also be used as biocontrol agents, for example, by acting antagonistically to various fungi that cause rot on harvested fruits and vegetables.

Example species of Rhodotorula and Rhodosporidium utilized in biotechnology include, but are not limited to, Rhodotorula aurantiaca, Rhodosporidium toruloides, Rhodotorula glutinis, Rhodotorula glutinis var. glutinis, Rhodotorula gracilis, Rhodosporidium diobovatum, Rhodotorula dairenensis, Rhodotorula diffluens, Rhodosporidium kratochvilovae, Rhodotorula graminis, Rhodotorula babjevae, Rodosporidium sphaerocarpum, Rhodotorula minuta, Rhodotorula mucilaginosa, Rhodotorula mucilaginosa, Rhodotorula terpenoidalis, Rhodotorula toruloides and Rhodotorula taiwanensis.

For example, R. toruloides is able to utilize multiple types of carbon for growth and production of high titers of lipids, which can then be used as biofuels, surfactants, solvents, and waxes (to name a few). R. toruloides was previously called Rhodotorula glutinis or Rhodotorula gracilis. R. glutinis is also able to produce lipids, and valuable enzymes, notably phenylalanine ammonia lyase (PAL), which is an important therapeutic enzyme with several medical applications, including phenylketonuria (PKU) treatment (Kawatra A., et al., Biomedical applications of microbial phenylalanine ammonia lyase: Current status and future prospects. 2020, Biochimie, 177:142-152). R. diobovatum may be used to produce glutathione in the near future, which is a valuable vitamin (Kong M., et al., Functional identification of glutamate cysteine ligase and glutathione synthetase in the marine yeast Rhodosporidium diobovatum (Naturwissenschaften. 2017 Dec. 15; 105(1-2):4). It's also being investigated as a biofuel production species (Valerie C. et al., ACS Sustainable Chemistry & Engineering 2017 5 (6), 5562-5570). R. kratochvilovae and R. graminis are also being used to create biofuels, and can produce carotenoids at high levels. Carotenoids have multiple uses, ranging from natural coloring agents in the food, cosmetic, and pharmaceutical industries, to antioxidants with protective health benefits. R. babjevae can produce polyol esters of fatty acids (PEFA), which are amphiphilic glycolipids that can be used as environmentally friendly biosurfactants (see for example WO2017184884A1). R. taiwanensis also produces biosurfactants, but with a different profile than that of R. babjevae that could have broader commercial applications.

### Methods of Extracting Bioproducts

The present disclosure teaches methods and compositions using orders of magnitude lesser quantities of enzyme than are reported in the literature, and further teach methods of solvent-free extraction of bioproducts.

In some embodiments, the disclosure relates to a method for isolating a bioproduct from a yeast comprising treating yeast cells with a β-1,3-glucomannanase, wherein the β-1,3-glucomannanase is in an amount of less than 1.0e-4 grams enzyme protein/gram dry cell weight, thereby producing an enzymatically lysed sample; separating the lipid phase from the aqueous phase of the enzymatically lysed sample via solvent or non-solvent extraction, thereby producing a separated sample; and isolating a bioproduct from the separated sample. In some embodiments, the bioproduct, e.g., a lipid or carotenoid, is contained in the lipid phase. In some embodiments, the bioproduct is isolated from the lipid phase. In some embodiments, the bioproduct, e.g., a saccharide, is contained in the aqueous phase. In some embodiments, the bioproduct is isolated from the aqueous phase.

In some embodiments, the yeast is an oleaginous yeast. In some aspects, the yeast is a species from the Rhodotorula, Rhodosporidium, or Sporobolomyces genus. In some aspects, the yeast is Rhodosporidium toruloides, Rhodotorula glutinis, Rhodosporidium diobovatum, Rhodosporidium kratochvilovae, Rhodotorula graminis, Rhodotorula babjevae, and Rhodotorula taiwanensis.

In some aspects, the β-1,3-glucomannanase is in an amount of less than 1.0e-5 grams enzyme protein/gram dry cell weight. In some aspects, the β-1,3-glucomannanase is in an amount of between 1.0e-6 and 5.0e-5 grams enzyme protein/gram dry cell weight. In some aspects, the β-1,3-glucomannanase is in an amount of less than 1.0e-6 grams enzyme protein/gram dry cell weight.

In some embodiments, the treating yeast cells with a β-1,3-glucomannanase occurs at between 20° C. and 55° C. In some aspects, the treating yeast cells with a β-1,3-glucomannanase occurs at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C.

In some embodiments, the yeast cells are treated with the β-1,3-glucomannanase for between 5 and 24 hours. In some aspects, the yeast cells are treated with the β-1,3-glucomannanase for about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some aspects, the yeast cells are treated with the β-1,3-glucomannanase for greater than 24 hours.

In some embodiments, the treating yeast cells with a β-1,3-glucomannanase occurs at a pH of between 4 and 5.5. In some aspects, the treating yeast cells with a β-1,3-glucomannanase occurs at a pH of about 4, about 4.1, about 4.2, about 4.3, about 4.4, about 4.5, about 4.6, about 4.7, about 4.8, about 4.9, about 5.0, about 5.1, about 5.2, about 5.3, about 5.4, or about 5.5.

In some embodiments, the separation is performed via solvent extraction. In some aspects, the solvent is hexane, heptane, ethanol, ethyl acetate, or chloroform and methanol. In some aspects, the chloroform:methanol ratio is 2:1. In some aspects, the solvent is not ethyl acetate.

In some embodiments, the solvent extraction is performed at between 30° C. and 55° C. In some aspects, the solvent extraction is performed at about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C.

In some embodiments, the solvent extraction is carried out for about 7-10 hours. In some embodiments, the solvent extraction is carried out for about 10-16 hours. In some aspects, the solvent extraction is carried out for about 7 hours, about 8 hours, about 9 hours, about 10 hours, about 11 hours, about 12 hours, about 13 hours, about 14 hours, about 15 hours, or about 16 hours. In some aspects, the solvent extraction is carried out for greater than 16 hours. In some embodiments, the solvent extraction is carried out for about 16-24 hours. In some embodiments, the solvent extraction is carried out for about 24-48 hours.

In some embodiments, a phospholipid solvent is added during extraction. In some aspects, the phospholipid solvent is ethanol, methanol, or acetone. In some aspects, the phospholipid solvent is ether, chloroform, or benzene.

In some embodiments, the yeast cells are treated with the β-1,3-glucomannanase and the solvent at the same time.

In some embodiments, the separation is carried out via non-solvent extraction. In some embodiments, the non-solvent extraction comprises gravimetric separation. In some embodiments, the method further comprises a mechanical treatment between the lysis and extraction. In some aspects, the mechanical treatment is at least one of bead milling, ultrasonication, high-pressure homogenization, shearing, and microwave irradiation. In some embodiments, the method further comprises an acid lysis. In some embodiments, the method comprises a physical pre-treatment of the yeast prior to treating with the β-1,3-glucomannanase. In some aspects, the physical pre-treatment is autoclaving, bead-milling, sonication, high-pressure homogenization, or microwave irradiation.

In some embodiments, the β-1,3-glucomannanase is an isolated and purified recombinant protein. In some aspects, β-1,3-glucomannanase is expressed and purified from Pichia pastoris. In some aspects, β-1,3-glucomannanase is expressed and purified from a recombinant microorganism transformed with an exogenous β-1,3-glucomannanase gene.

### Solvent-Free Extraction of Bioproducts

In some embodiments, the disclosure teaches a method for enzymatic lysis of microorganisms having recalcitrant cell walls comprising: inactivating a biomass of microorganisms having recalcitrant cell walls, inoculating the inactive biomass with live cellulolytic fungi and/or an organism engineered to express at least one cellulolytic enzyme, and incubating the live cellulolytic fungi and/or an organism engineered to express at least one cellulolytic enzyme for at least 5 hours to generate a lysed biomass.

In some embodiments, the inactive biomass to live cell ratio is between 10,000:1 and 1:1 dry cell w/w. In some aspects, the inactive biomass to live cell ratio is between 10,000:1 and 10:1 dry cell w/w. In some embodiments, the inactive biomass to live cell ratio is between 10,000:1 and 100:1 dry cell w/w.

In some embodiments, the incubating is for at least 10 hours. In some aspects, the incubating is for at least 15 hours, at least 20 hours, at least 25 hours, at least 30 hours, at least 35 hours, or at least 40 hours. In some embodiments, the incubating is for at least 4 days, 5 days, 6 days, 7 days, 8 days, or 9 days.

In some embodiments, the incubating occurs at between 20° C. and 55° C. In some aspects, the incubating occurs at about 20° C. about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C.

### Cellulolytic Fungi

In some embodiments, the cellulolytic enzyme and/or β-1,3-glucomannanase is produced and/or secreted by a cellulolytic fungi. In some embodiments, the cellulolytic fungi is a species of Trichoderma, Humicola, Purpureocillium, Penicillium, Phanerochaete, or Pycnoporus.

In some aspects, the cellulolytic fungi is a species of Trichoderma. In some aspects, the species is T. reesei, T. longibrachiatum, T. atroviride, T. virens, T. viride, T. hamatum, or T. harzianum. See also Do Vale L. H. F. et al., Cellulase Systems in Trichoderma: An Overview, 2014. In some aspects, the species of Trichoderma has been genetically modified to express, overexpress, and/or secrete an enzyme.

In some aspects, the cellulolytic fungi is a species of Humicola. In some aspects, the species is H. alopallonella, H. ampulliella, or H. asteroidea. See also Wang, X. W. et al., Redefining Humicola sensu stricto and related genera in the Chaetomiaceae, 2019, Studies in Mycology 93:65-153. In some aspects, the species of Humicola has been genetically modified to express, overexpress, and/or secrete an enzyme.

In some aspects, the cellulolytic fungi is a species of Purpureocillium. In some aspects, the species is P. atypicola, P. lavendulum P. lilacinum, P. sodanum, or P. takamizusanense. In some aspects, the species of Purpureocillium has been genetically modified to express, overexpress, and/or secrete an enzyme.

In some aspects, the cellulolytic fungi is Penicillium sp., P. camemberti, P. citrinum, P. griseoroseum, P. restrictum or P. roqueforti. In some aspects, the species of Penicillium has been genetically modified to express, overexpress, and/or secrete an enzyme.

In some aspects, the cellulolytic fungi is a species of Phanerochaete. In some aspects, the species is Phanerochaete sp., P. velutina, or P. chrysosporium. See also Floudas, D, and Hlibbett, D. S. (2015) “Revisiting the taxonomy of Phanerochaete (Polyporales, Basidiomycota) using a four gene dataset and extensive ITS sampling”. Fungal Biolog, 119: 679-719. In some aspects, the species of Phanerochaete has been genetically modified to express, overexpress, and/or secrete an enzyme.

In some aspects, the cellulolytic fungi is a species of Pycnoporus. In some aspects, the species is P. cinnabarinus, P. coccineus, P. palibini, P. puniceus, or P. sanguineus. See also Lomascolo, A., et al., (2011). “Peculiarities of Pycnoporus species for applications in biotechnology”. Applied Microbiology and Biotechnology. 92 (6): 1129-1149. In some aspects, the species of Pycnoporus has been genetically modified to express, overexpress, and/or secrete an enzyme.

In some embodiments, the cellulolytic fungi has been genetically modified to produce and/or secrete β-1,3-glucomannanase. In some aspects, the modified cellulolytic fungi is Purpureocillium lilacinum. In some aspects, the modified cellulolytic fungi is Trichoderma reesei.

In some embodiments, the cellulolytic enzyme and/or β-1,3-glucomannanase is produced and/or secreted by a recombinant microorganism transformed to express an exogenous cellulotyic enzyme gene and/or β-1,3-glucomannanase gene from a cellulolytic fungi.

### Additional Enzymes

In some embodiments, the methods of the present disclosure further comprises a second or more enzyme. In some embodiments, the second or more enzyme is a protease. In some embodiments, the second or more enzyme is a cellulase. In some embodiments, the second or more enzyme is a phospholipase. In some embodiments, the second or more enzyme is a glycosyl-hydrolase. In some embodiments, the second or more enzyme is an oxidoreductase. In some embodiments, the second or more enzyme is a lyase. In some embodiments, the second or more enzyme is an esterase.

In some embodiments, the protease is an aminopeptidase or carboxypeptidase. In some aspects, the carboxypeptidase is a serine peptidase, metallopeptidase, or cysteine peptidase. In some embodiments, the protease is an endopeptidase. In some aspects, the endopeptidase is a serine protease, cysteine protease, aspartic protease, or metalloprotease.

In some embodiments, the second or more enzyme is a xylanase, galactosidase, glucuronidase, cellobiohydrolase, endoglucanase, lactase, mannanase, and/or pectinase.

In some embodiments, the β-1,3-glucomannanase is a part of an enzyme cocktail comprising two or more enzymes. In some embodiments, the β-1,3-glucomannanase is comprised within a blended enzyme extract from two or more microorganisms. In some embodiments, a second or more enzyme is added prior to, or during extraction.

### Engineered Microbes

In some embodiments, the disclosure relates to engineering a microorganism to produce one or more cellulolytic enzymes. In some aspects, the modification may be increasing expression of an existing (endogenous) enzyme(s). In some aspects, the modification may be inserting one or more heterologous cellulolytic genes. In some aspects, a constitutive, inducible, or repressible promoter is inserted upstream of the one or more cellulolytic enzyme genes.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages.

As used herein, “inducible” or “repressible” promoter is a promoter that is under chemical or environmental factor's control. Examples of environmental conditions that may affect transcription by inducible promoters include anaerobic conditions, certain chemicals, the presence of light, acidic or basic conditions, etc.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

Example promoters for use are well known in the art. See for example Liu, Y., et al. Engineering an efficient and tight D-amino acid-inducible gene expression system in Rhodosporidium Rhodotorula species. Microb Cell Fact 14, 170 (2015); Liu Y, et al., Developing a set of strong intronic promoters for robust metabolic engineering in oleaginous Rhodotorula (Rhodosporidium) yeast species. Microb Cell Fact. 2016 Nov. 25; 15(1):200; Nora L C, et al., A toolset of constitutive promoters for metabolic engineering of Rhodosporidium toruloides. Microb Cell Fact. 2019 Jun. 29; 18(1):117; Johns, A. M. B., et al., Four Inducible Promoters for Controlled Gene Expression in the Oleaginous Yeast Rhodotorula toruloides. Frontiers in Microbiology, October 2016; Pi, H W., et al. Engineering the oleaginous red yeast Rhodotorula glutinis for simultaneous β-carotene and cellulase production. Sci Rep 8, 10850 (2018).

Examples of promoters that may be used to engineer organisms to express a particular bioproduct or a cellulolytic enzyme include, but are not limited, to those listed below in Table 1. Accession numbers are to either the UniProt or Rhodosporidium toruloides IF00880 v4.0 genome (Protein ID and Transcript ID), which can be found on the Joint Genome Institute (JGI) MycoCosm site on the world wide web at: genome.jgi.doe.gov/Rhoto_IF00880_4/Rhoto_IF00880_4.home.html, unless otherwise noted.

TABLE 1
Example promoters

| Promotor of gene: | UniProtKB or other ID | |
|---|---|---|
| Arabinose inducible promoter LADp | | |
| Tetracycline resistance protein (tetO) | P20174 | |
| Alcohol oxidase 1 (AOX1) | P04842 | |
| Phosphoadenosine phosphosulfate reductase (MET16) | P18408 | |
| Copper transport protein (CTR3) | Q06686 | |
| Isocitrate lyase (ICL1) | P28240 | |
| Cytosolic Fe-S cluster assembly factor (NAR1) | P23503 | |
| Fructose-bisphosphate aldolase (FBA1) | P14540 | |
| Phosphoglycerate kinase (PGK1) | P00560 | |
| Triosephosphate isomerase (TPI1) | P00942 | |
| Phospholipid:diacylglycerol acyltransferase (LRO) | P40345 | |
| Diacylglycerol O-acyltransferase 1 (DGA1) | Q08650 | |
| Orotate phosphoribosyltransferase 1 (URA5) | P13298 | |
| Orotidine 5′-phosphate decarboxylase (URA3) | P03962 | |
| Glucose-6-phosphate isomerase (PGI1) | P12709 | |
| 3-isopropylmalate dehydrogenase (LEU2) | P04173 | |
| Xylose reductase (XYL1) | I7B253 | |
| Elongation factor 1-alpha (TEF1) | P02994 | |
| ATP-citrate synthase subunit 1 (ACL1) | 093988 | |
| Very long-chain fatty acid transport protein (FAT1) | P38225 | |
| Urea amidolyase (DUR1, 2) | P32528 | |
| D-amino acid oxidase (DAO1) | P80324 | |
| Perilipin/lipid droplet protein 1 (LDP1) | RHTO-05627* | |
| Glyceraldehyde-3-phosphate dehydrogenase (GPD1) | Q00055 | |
| Acetyl-CoA carboxylase (ACC1) | Q00955 | |
| Fatty acid synthase β subunit (FAS1) | P07149 | |
| | Protein ID | Transcript ID |
| K02984: RP-S3Ae, RPS3A; small subunit ribosomal protein S3Ae | 10325 | 10453 |
| 40S ribosomal protein S1 [Exophiala sideris] | 10324 | 10452 |
| K04564: SOD2; superoxide dismutase, Fe—Mn family | 15909 | 16037 |
| K01012: bioB; biotin synthase | 15908 | 16036 |
| K15040: VDAC2; voltage-dependent anion channel protein 2 | 11021 | 11149 |
| K02925: RP-L3e, RPL3; large subunit ribosomal protein L3e | 14514 | 14642 |
| K02969: RP-S20e, RPS20; small subunit ribosomal protein S20e | 10055 | 10183 |
| Domain of Unknown Function | 15265 | 15393 |
| K00134: GAPDH, gapA; glyceraldehyde 3-phosphate dehydrogenase | 10613 | 10741 |
| KOG0004: Ubiquitin/40S ribosomal protein S27a fusion | 8752 | 8880 |
| K02922: RP-L37e, RPL37; large subunit ribosomal protein L37e | 8751 | 8879 |
| K11254: H4; histone H4 | 9231 | 9359 |
| K11253: H3; histone H3 | 9232 | 9360 |
| K08770: UBC; ubiquitin C | 13099 | 13227 |
| K02979: RP-S28e, RPS28; small subunit ribosomal protein S28e | 14295 | 14423 |
| K02989: RP-S5e, RPS5; small subunit ribosomal protein S5e | 14294 | 14422 |
| K02979: RP-S28e, RPS28; small subunit ribosomal protein S28e | 14295 | 14423 |
| K02989: RP-S5e, RPS5; small subunit ribosomal protein S5e | 14294 | 14422 |
| K02947: RP-S10e, RPS10; small subunit ribosomal protein S10e | 9006 | 9134 |
| K18027: PTPN3, PTPH1; tyrosine-protein phosphatase non-receptor | 9007 | 9135 |
| type 3 | | |
| K03231: EEF1A; elongation factor 1-alpha | 12693 | 12821 |
| K05863: SLC25A4S, ANT; solute carrier family 25 (mitochondrial | 12704 | 12832 |
| adenine nucleotide translocator), member 4/5/6/31 | | |
| K02978: RP-S27e, RPS27; small subunit ribosomal protein S27e | 14937 | 15065 |
| Hypothetical protein | 15825 | 15953 |
| K01647: CS, gltA; citrate synthase | 11331 | 11459 |
| K02958: RP-S15e, RPS15; small subunit ribosomal protein S15e | 16419 | 16547 |
| K02943: RP-LP2, RPLP2; large subunit ribosomal protein LP2 | 16418 | 16546 |
| K03768: PPIB, ppiB; peptidyl-prolyl cis-trans isomerase B | 8813 | 8941 |
| (cyclophilin B) | | |
| K03094: SKP1, CBF3D; S-phase kinase-associated protein 1 | 9048 | 9176 |
| Hypothetical protein | 9047 | 9175 |
| KOG1817: Ribonuclease | 12216 | 12344 |
| Hypothetical protein | 12215 | 12343 |
| HMMPfam: Domain of unknown function (DUF543):PF04418 | 12844 | 12972 |
| KOG3469: Cytochrome c oxidase, subunit VIa/COX13 | 13007 | 13135 |
| HMMPfam: GDSL/SGNH-like Acyl-Esterase family found in Pmr5 | 13006 | 13134 |
| and Caslp: PF13839, SUPERFAMILY::SSF52266 | | |
| HMMPfam: Ubiquinol-cytochrome-c reductase complex subunit | 13608 | 13736 |
| (QCR10): PF09796 | | |
| K10859: ALKBH2; alpha-ketoglutarate-dependent dioxygenase alkB | 13607 | 13735 |
| homolog 2 | | |
| K00411: UQCRFS1, RIP1, petA; ubiquinol-cytochrome c reductase | 13614 | 13742 |
| iron-sulfur subunit | | |
| K17279: REEP5_6; receptor expression-enhancing protein 5/6 | 14603 | 14731 |
| K12845: SNU13, NHP2L; U4/U6 small nuclear ribonucleoprotein | 15467 | 15595 |
| SNU13 | | |
| KOG2633: Hismacro and SEC14 domain-containing proteins | 15466 | 15594 |
| K04393: CDC42; cell division control protein 42 | 15484 | 15612 |
| K12868: SYF2; pre-mRNA-splicing factor SYF2 | 15485 | 15613 |

*Genbank number

Terminators are also well known in the art, and include, but are not limited to, SV40, 35S (see for example Liu Y., et al., Characterization of glyceraldehyde-3-phosphate dehydrogenase gene RtGPD1 and development of genetic transformation method by dominant selection in oleaginous yeast Rhodosporidium toruloides. Appl Microbiol Biotechnol 2013; 97:719-29; Koh C M J et al., Molecular characterization of KU70 and KU80 homologues and exploitation of a KU70-deficient mutant for improving gene deletion frequency in Rhodosporidium toruloides. BMC Microbiol 2014; 14:50; Liu Y B, et al., Engineering an efficient and tight D-amino acid-inducible gene expression system in Rhodosporidium Rhodotorula species. Microb Cell Fact 2015; 14:170), NOS, hsp, (see for example Lin et al., Functional integration of multiple genes into the genome of the oleaginous yeast Rhodosporidium toruloides. FEMS Yeast Res 2014; 14:547-55; Wang Y, et al. Overexpression of Δ12-fatty acid desaturase in the oleaginous yeast Rhodosporidium toruloides for production of linoleic acid-rich lipids. Appl Biochem Biotechnol 2016; 180:1497-507), URA5, URA3, INO, LRO, DGA (see for example Lin X, et al., Functional integration of multiple genes into the genome of the oleaginous yeast Rhodosporidium toruloides. FEMS Yeast Res 2014; 14:547-55), etc.

In some embodiments, the disclosure relates to modifying a microorganism to express and secrete a cellulolytic enzyme. In some embodiments, disclosure relates to engineered autolytic industrious yeast that produce a bioproduct, and methods of making the same. In some aspects, the autolytic industrious yeast comprises a heterologous cellulolytic enzyme gene under the control of an inducible promoter. In some aspects, the gene is MAN5C from Purpureocillium lilacinum. In some aspects, the yeast is Rhodosporidium toruloides.

Yeasts possess many of the post-translational and secretion pathways present in higher eukaryotes, and are highly amenable to genetic modifications. For example, protein secretion may be increased by targeted modifications to the secretory and trafficking genes and pathways (Huang, M. et al., Engineering the protein secretory pathway of Saccharomyces cerevisiae enables improved protein production, 2018 PNAS 115 (47); Huang, M. et al., Microfluidic screening and whole-genome sequencing identifies mutations associated with improved protein secretion by yeast, 2015 PNAS 112 (34); de Ruijter, et al., (2016)). Enhancing antibody folding and secretion by tailoring the Saccharomyces cerevisiae endoplasmic reticulum Microb. Cell Fact 15, 1-18).

Transformation methods are well known in the art and include, for example, PEG-mediated protoplast transformation (Gilbert, 1985), ATMT random insertion (Liu et al. 2013; Lin et al. 2014), ATMT targeted deletion (Sun et al., Homologous gene targeting of a carotenoids biosynthetic gene in Rhodosporidium toruloides by Agrobacterium-mediated transformation. Biotechnol Lett 2017, 39:1001-7; Koh et al. 2014), LiAc/PEG-mediated chemical transformation (random insertion) (Tsai et al., Development of a sufficient and effective procedure for transformation of an oleaginous yeast, Rhodosporidium toruloides DMKU3-TK16. Curr Genet 2017; 63:359-71), and electrotransformation (Takahashi S et al., Genetic transformation of the yeast Rhodotorula gracilis ATCC 26217 by electroporation. Appl Biochem Microbiol 2014; 50:624-8; Liu et al., Fast and efficient genetic transformation of oleaginous yeast Rhodosporidium toruloides by using electroporation. ELMS Yeast Res 2017; 17).

Example Yeasts for Use with the Disclosed Methods and Compositions

Table 2 shows some example Rhodosporidium and Rhodotorula species and strains that produce bioproducts which could be used with the methods and compositions disclosed herein.

TABLE 2
Example industrious yeast strains (partially adapted from Zhiqiang
Wen, et al., FEMS Yeast Research, Volume 20, Issue 5, August 2020)

| Strain | | | |
|---|---|---|---|
| engineering or | Species and | | |
| optimization | designation | Description | References |
| Product | Rhodosporidium | Integration of the gene encoding a | Lee et al. 2016 |
| export/secretion | toruloides | membrane transporter, pleiotropic | |
| engineering | CBS 5490 | drug resistance (Pdr) 10 from S. | |
| | | cerevisiae. Carotenoid efflux | |
| | | efficiency increased by 5-fold; | |
| | | Concurrent carotenoids/intracellular | |
| | | lipids separation; Carotenoid yield | |
| | | increase from 1.9 to 2.9 μg/mg. | |
| Lipid | R. toruloides | Developing a new acetyl-CoA | Yang et al 2018 |
| production | NP11 | synthesis pathway via integration of | |
| promotion | | the gene encoding | |
| | | phosphotransacetylase from | |
| | | Bacillus subtilis. Cell mass, lipid | |
| | | yield and lipid productivity | |
| | | enhanced by 26%, 11% and 54%, | |
| | | respectively. | |
| | R. toruloides | Random integration of genes | Diaz et al. 2018 |
| | CECT 13085 | encoding endogenous DGA1 and | |
| | | SCD1 via ATMT; Lipid yield | |
| | | increased by 13.3%; lipid titer | |
| | | reached 39.5 g/L from undetoxified | |
| | | lignocellulosic hydrolysates. | |
| | R. toruloides | Random integration of genes | Zhang et al. 2016 |
| | IFO 0880 | encoding endogenous ACCI and | |
| | | DGA1 via ATMT. Lipid titer | |
| | | increased by 74.6% to 24.8 g/L; | |
| | | lipid titer increased by 2.49-fold to | |
| | | 62.8 g/L in a fed-batch culture. | |
| | R. toruloides | Random integration of genes | Zhang et al. 2016 |
| | IFO 0880 | encoding endogenous ACC1, | |
| | | DGA1 and SCD1 genes via ATMT. | |
| | | Lipid titer increased by 85.1% to | |
| | | 27.4 g/L; lipid titer increased by | |
| | | 3.97-fold to 89.4 g/L in a fed-batch | |
| | | culture. | |
| | R. toruloides | UV mutagenesis plus orientation | Yamada et al. 2017 |
| | NBRC 8766 | screening. Lipid output and | |
| | | productivity increased significantly; | |
| | | transcriptional analysis revealed | |
| | | dramatic up-regulation of IDP1 and | |
| | | ME1 gene. | |
| | R. toruloides | Atmospheric and room temperature | Zhang et al. 2016 |
| | NP11 | plasma (ARTP) and chemical | |
| | | mutagenesis. Lipid and carotenoid | |
| | | production improved simultaneously. | |
| | R. toruloides | Random integration of the gene | Wang et al. 2016 |
| | AS 2.1389 | encoding endogenous malic enzyme | |
| | | via ATMT. Lipid content and yield | |
| | | increased by 84% and 91%, | |
| | | respectively, under phosphorus- | |
| | | limited conditions. | |
| Diversity | R. toruloides | Random integration of the genes | Wang et al. 2016 |
| exploration of | AS 2.1389 | encoding Δ12-fatty acid desaturase | |
| fatty acids | | from Fusarium verticillioides. | |
| derived- | | Linoleic acid content increased by | |
| product | | 5-fold to a final linoleic acid titer of | |
| | | 1.3 g/L under flask culture | |
| | | conditions. | |
| | R. toruloides | Random integration of genes | Fillet et al. 2016 |
| | CECT 13085 | encoding Δ12 desaturase and ω-3 | |
| | | desaturase with in-frame deletion of | |
| | | aldehyde dehydrogenase via | |
| | | ATMT. α-Linolenic acid content | |
| | | reached ~49% of total fatty acids. | |
| | R. toruloides | Random integration of genes | Fillet et al. 2017 |
| | CECT 13085 | encoding 3-ketoacyl-CoA synthase | |
| | | from different plants. Produced very | |
| | | long-chain fatty acids erucic acid | |
| | | and nervonic acid at final titers of | |
| | | 5.8 g/L and 7.9 g/L, respectively. | |
| | R. toruloides | Random integration of the gene | Fillet et al. 2015 |
| | CECT 13085 | encoding fatty acyl-CoA reductase | |
| | | via ATMT. Long chain (C16-C18) | |
| | | fatty alcohols were secreted | |
| | | extracellularly with final titers up to | |
| | | 8.0 g/L. | |
| | R. toruloides | Random integration of genes | Tsai et al. 2019 |
| | NP11 | encoding ScOle1 and the | |
| | R. toruloides | homologue RtΔ9Fad via LiAc/PEG- | |
| | TK16 | mediated chemical transformation. | |
| | | Produced lipids with 5-fold more | |
| | | oleic acid content. | |
| Metabolic | R. toruloides | Random integration of genes | Zhuang et al. 2019 |
| engineering for | IFO 0880 | encoding terpene synthases via | |
| | | ATMT. Produced 1,8-cineole at up | |
| | | to 34.6 mg/L from biomass | |
| | | hydrolysates. | |
| production of | R. toruloides | Random integration of genes | Yaegashi et al. |
| terpenoids | IFO 0880 | encoding bisabolene synthase and | 2017; Sundstrom |
| | | amorphadiene synthase via ATMT. | et al. 2018; |
| | | Produced bisabolene at 680 mg/L | Pimienta et al. |
| | | on the alkaline corn stover | 2019 |
| | | hydrolysates; bisabolene titers | |
| | | improved to 2.2 g/L in 20-L | |
| | | bioreactor, using separation-free | |
| | | biomass hydrolysates as substrate. | |
| Metabolic | R. toruloides | Random integration of genes | Wehrs et al. 2019 |
| engineering for | IFO 0880 | encoding BpsA and sfp via ATMT. | |
| non-ribosomal | | Produced indigoidine at up to 2.9 ± | |
| peptides | | 0.8 g/L using a sorghum biomass | |
| production | | hydrolysates in a batch process and | |
| | | 86.3 ± 7.4 g/L using glucose in a | |
| | | high-gravity fed-batch process. | |
| Stress- | R. toruloides | Atmospheric and room temperature | Qi et al. 2014 |
| resistance | ACCC 20341 | plasma mutagenesis. Obtain mutant | |
| improvement | | strain with higher tolerance to | |
| | | biomass hydrolysates inhibitor; both | |
| | | cell mass and lipid content | |
| | | increased by over 10%. | |
| Optimization | Rhodotorula | Different methods of enriching PAL | D'Cunha G.B.D., |
| for | glutinis | activity | 2005 |
| Phenylalanine | NCYC 61 | | |
| ammonia lyase | | | |
| production | | | |
| Metabolic | Rhodosporidium | | Kong et al. 2017 |
| engineering for | diobavatum | | |
| production of | | | |
| glutathione | | | |
| Metabolic | R. diobavatum | | Valerie et al. 2017 |
| engineering for | | | |
| biofuel | | | |
| production | | | |
| Metabolic | Rhodotorula | | |
| engineering for | kratochvilovae | | |
| biofuel and | | | |
| carotenoids | | | |
| production | | | |
| Metabolic | Rhodotorula | | |
| engineering for | graminis | | |
| biofuel and | | | |
| carotenoids | | | |
| production | | | |
| Optimization | Rhodotorula | Yeast cultures for production of | WO2017184884A1 |
| for production | babjevae | polyol esters of fatty acids | |
| of polyol esters | UCDFST 04-877 | | |
| of fatty acids | NRRL Y-67018 | | |
| | NRRL Y-67017 | | |
| | UCDFST 68- 916.1 | | |
| | UCDFST 67-458 | | |
| | UCDFST 05-736 | | |
| | UCDFST 04-830 | | |
| Optimization | R. diobovata | Yeast cultures for production of | WO2017184884A1 |
| for production | NRRL Y-67015 | polyol esters of fatty acids | |
| of polyol esters | UCDFST 04-830 | | |
| of fatty acids | | | |
| Optimization | R. kratochvilovae | Yeast cultures for production of | WO2017184884A1 |
| for production | NRRL Y-67016 | polyol esters of fatty acids | |
| of polyol esters | | | |
| of fatty acids | | | |
| Optimization | Rhodotorula | Yeast cultures for production of | WO2017184884A1 |
| for production | paludigena | polyol esters of fatty acids | |
| of polyol esters | NRRL Y-67012 | | |
| of fatty acids | UCDFST 81-492 | | |
| | UCDFST 82-646.2 | | |
| | NRRL Y-67009 | | |
| Optimization | Rhodotorula | Yeast cultures for production of | WO2017184884A1 |
| for production | dairenensis | polyol esters of fatty acids | |
| of polyol esters | NRRL Y-67011 | | |
| of fatty acids | | | |
| Environmental | Rhodotorula | Produces biosurfactants composed | Lyman et al., 2018 |
| isolate for | taiwanensis | of PEFA compounds composed | |
| production of | MD1149 | mainly of mannitol and arabitol | |
| surfactants | | esters of 3-hydroxy fatty acid, 3- | |
| | | methoxy fatty acid, and fatty acids | |
| | | with a single double bond; chain | |
| | | lengths are mainly C16 and C18. | |
| | | PEFA compounds are more surface | |
| | | active due to their hypoacetylation | |
| | | profile (0-4 acetylation | |
| | | modifications) compared to | |
| | | Rhodotorula babjevae MD1169. | |
| Environmental | Rhodosporidiobolus | Produces extracellular | Tulloch et al. 1964 |
| isolate from | azoricus | glycolipids which consist of a | |
| Canada | CBS 4648 | mixture of mannitol and pentitol | |
| | | esters of 3-D-hydroxypalmitic | |
| | | and 3-D-hydroxystearic acids. | |
| Environmental | Rhodotorula | Produces extracellular | Tulloch et al. 1964 |
| isolate from | glutinis | glycolipids which consist of a | |
| Canada | 16A8 | mixture of mannitol and pentitol | |
| | | esters of 3-D-hydroxypalmitic | |
| | | and 3-D-hydroxystearic acids. | |
| Environmental | R. graminis | Similar to CBS 4648 and 16A8 | Tulloch et al. 1964 |
| isolate from | 6CB | above but contains different relative | |
| Canada | | amounts of palmitic and oleic acids. | |
| Optimized for | R. toruloides | Produced 66% w/w dry wt lipids | Johnson et al. 1992 |
| lipid production | IIP-30 | utilizing glucose in a fed-batch | |
| | | fermentation under N-limiting | |
| | | conditions at 30° C., pH 4. | |
| Optimized for | R. babjevae | Secretes extracellular glycolipids | Cajka, 2016 |
| lipid production | UCDFST 04-877 | with biosurfactant properties. | |
| New isolate for | R. babjevae | Accumulates high intracellular | Guerfali et al., |
| production of | Y-SL7 | content of microbial oil (mainly | 2019 |
| glycolipids | | triacylglycerols) and secrets, under | |
| | | the same culture conditions, an | |
| | | atypical glycolipid which is | |
| | | therapeutically promising as a | |
| | | cytotoxic effect against different | |
| | | cancer cell lines. | |
| New deep-sea | R. paludigena | Produces high level of intracellular | Wang et al. 2019 |
| isolate | P4R5 | lipid and extracellular PEFA. Under | |
| candidate for | | the optimized conditions, can yield | |
| biosurfactant | | 48.5 g/L of PEFA and 16.9 g/L of | |
| and biodiesel | | intracellular lipid within 156 h from | |
| production | | inulin during 10-L batch | |
| | | fermentation. | |
| | R. toruloides | Produces lipid compositions that | WO2021/163194 |
| | strain A, strain B, | can be used as alternatives to palm | WO2021/154863 |
| | and strain C | oil | |
| | R. toruloides | | |
| | CBS 6016 | | |

In some embodiments, the yeast is of the genus Rhodotorula. In some embodiments, the yeast is of the genus Rhodosporidium. In some embodiments, the yeast is of the genus Sporobolomyces.

In some embodiments, the methods of the present disclosure relate to homogeneous populations comprising microorganisms of the same species and strain. In some embodiments, the methods of the present disclosure relate to a heterogeneous population comprising microorganisms from more than one species and/or strain.

### Pigments

In some embodiments, the bioproduct comprises a pigment. In some embodiments, the bioproduct comprises at least one pigment selected from the group consisting of carotene, torulene and torulorhodin.

In some embodiments, the bioproduct comprises carotene. In some embodiments, the bioproduct comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ppm, or any ranges or subranges therebetween, of carotene. In some embodiments, the bioproduct comprises at least 25 ppm of carotene. In some embodiments, the bioproduct comprises at least 50 ppm of carotene. In some embodiments, the bioproduct comprises at least 100 ppm of carotene. In some embodiments, the carotene is β-carotene and/or a derivative thereof. In some embodiments, the carotene is (13Z)-β-Carotene. In some embodiments, the carotene is (9Z)-β-Carotene.

In some embodiments, the bioproduct comprises torulene. In some embodiments, the bioproduct comprises torulorhodin. In some embodiments, the bioproduct comprises a derivative of torulene and/or torulorhodin. In some embodiments, the bioproduct comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ppm, or any ranges or subranges therebetween, of torulene, torulorhodin, and/or derivatives thereof. In some embodiments, the bioproduct comprises at least 25 ppm of torulene, torulorhodin, and/or derivatives thereof. In some embodiments, the bioproduct comprises at least 50 ppm of torulene, torulorhodin, and/or derivatives thereof. In some embodiments, the bioproduct comprises at least 100 ppm of torulene, torulorhodin, and/or derivatives thereof. In some embodiments, the bioproduct comprises at least 300 ppm of torulene, torulorhodin, and/or derivatives thereof.

In some embodiments, the bioproduct comprises each of carotene and torulene.

### Compositions

In some embodiments, the disclosure relates to compositions comprising two or more enzymes, wherein the two or more enzymes comprise an isolated and purified β-1,3-glucomannanase and at least one of a cellulase and a protease; and an inactive cell biomass, wherein the composition has a total enzyme to dry cell weight ratio between 1:10,000 and 1:1,000,000.

In some aspects, the enzyme to dry cell weight ratio is between 1:10,000 and 1:100,000.

In some embodiments, the inactive cell biomass comprises one or more species of Rhodotorula, Rhodosporidium, or Sporobolomyces.

In some embodiments, the disclosure relates to a composition comprising a live cellulolytic fungus and an inactive yeast, wherein the cellulolytic fungus is a species selected from Trichoderma, Humicola, Penicillium, Purpureocillium, Phanerochaete, and Pycnoporus, and wherein the inactive yeast is a species selected from Rhodotorula, Rhodosporidium, or Sporobolomyces, and wherein the inactive yeast to live cellulolytic fungus ratio is between 1000:1 and 1:1 dry cell w/w.

In some aspects, the cellulolytic fungus produces β-1,3-glucomannanase. In some aspects, the cellulolytic fungus has been genetically engineered to produce 1,3-glucomannanase. In some aspects, the cellulolytic fungus is Purpureocillium lilacinum and the inactive yeast is Rhodosporidium toruloides.

In some embodiments, the inactive yeast has been genetically modified to produce a bioproduct.

In some embodiments, the inactive yeast to live cellulolytic fungus ratio is between 1000:1 and 10:1 dry cell w/w. In some aspects, the inactive yeast to live cellulolytic fungus ratio is between 1000:1 and 100:1 dry cell w/w.

### Bioproducts

In some embodiments, the methods of the present disclosure further comprise isolating a bioproduct from the lysed biomass or composition. In some embodiments, bioproduct is a lipid, carotenoid, protein, saccharide, or combination thereof.

In some embodiments, the disclosure relates to bioproducts comprising less than 10 ppm of a solvent. In some aspects, the bioproduct comprises less than 8 ppm, less than 6 ppm, less than 4 ppm, or less than 2 ppm of a solvent. In some aspects, the bioproduct does not comprise a detectable amount of solvent. In some aspects, the solvent is heptane, hexane, ethyl acetate, ethanol, chloroform, and/or methanol. In some embodiments, the bioproduct comprises at least one pigment. In some aspects, the pigment is selected from carotene, torulene and torulorhodin.

### Lipids

In some embodiments, the bioproduct is a lipid, e.g., a microbial oil produced from an oleaginous yeast. In some embodiments, the lipids are isolated by gravimetric separation. In some embodiments, the microbial oil comprises a fatty acid profile comprising at least 30% w/w saturated fatty acids, at least 30% w/w unsaturated fatty acids, and less than 30% w/w total polyunsaturated fatty acids. In some aspects, the microbial oil comprises a fatty acid profile comprising greater than 40% w/w saturated fatty acids, greater than 40% w/w mono-unsaturated fatty acids, and less than 20% w/w polyunsaturated fatty acids.

In some aspects, the microbial oil comprises β-carotene and torulene. In some aspects, the microbial oil comprises at least 10 ppm, at least 50 ppm, or at least 100 ppm torulene.

In some embodiments, the microbial oil comprises at least 5%, at least 10%, at least 15%, at least 20%, at least 25%, at least 30%, at least 35%, at least 40%, at least 45%, at least 50%, at least 55%, or at least 60% w/w palmitic acid (C16:0), or any ranges or subranges therebetween. In some embodiments, the microbial oil comprises at least 5% w/w palmitic acid. In some embodiments, the microbial oil comprises at least 10% w/w palmitic acid. In some embodiments the microbial oil comprises about 10-40% w/w palmitic acid. In some embodiments the microbial oil comprises about 13-35% w/w palmitic acid.

In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9% or at least 10% w/w palmitoleic acid (C16:1), or any ranges or subranges therebetween. In some embodiments, the microbial oil comprises at least 0.1% w/w palmitoleic acid. In some embodiments, the microbial oil comprises at least 0.5% w/w palmitoleic acid. In some embodiments, the microbial oil comprises about 0.5-10% w/w palmitoleic acid. In some embodiments, the microbial oil comprises about 0.5-5% w/w palmitoleic acid.

In some embodiments, the microbial oil comprises margaric acid (C17:0). In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% margaric acid, or any ranges or subranges therebetween. In some embodiments, the microbial oil comprises about 5-25% w/w margaric acid. In some embodiments, the microbial oil comprises about 9-21% w/w margaric acid.

In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% w/w stearic acid (C18:0), or any ranges or subranges therebetween. In some embodiments, the microbial oil comprises between about 7.0 and 35% w/w stearic acid. In some embodiments, the microbial oil comprises about 9-21% w/w stearic acid.

In some embodiments, the microbial oil comprises at least 5%, at least 10%, at least 15%, at least 20%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54% at least 55%, at least 56%, at least 57%, at least 58%, at least 59%, or at least 60% w/w oleic acid (C18:1), or any ranges or subranges therebetween. In some embodiments, the microbial oil comprises at least 25% w/w oleic acid. In some embodiments, the microbial oil comprises at least 30% w/w oleic acid. In some embodiments, the microbial oil comprises about 30-65% w/w oleic acid. In some embodiments, the microbial oil comprises about 39-55% w/w oleic acid. In some embodiments, the microbial oil comprises between about 10% and 50% w/w oleic acid.

In some embodiments, the microbial oil comprises C18:2 (linoleic acid). In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, or any ranges or subranges therebetween. In some embodiments, the microbial oil comprises about 5-25% linoleic acid. In some embodiments, the microbial oil comprises about 8-20% linoleic acid.

In some embodiments, the microbial oil comprises C18:3 (linolenic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linolenic acid, or any ranges or subranges therebetween.

In some embodiments, the microbial oil comprises C20:0 (arachidic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% arachidic acid, or any ranges or subranges therebetween.

### Carotenoids

In some embodiments, the bioproduct is a carotenoid. Carotenoids are a group of lipid-soluble yellow to red pigments with many valuable properties and uses, such as antioxidants and vitamin A activity. They are of use in the food—both for the health benefits and as alternatives for artificial pigments, animal feed, dietary supplements, personal care and cosmetics, and pharmaceutical industries. The global market for carotenoids in 2022 (based on data from 2021) is projected to be USD 2.0 billion and is expected to reach 2.7 billion by 2027. β-carotene, produced by the red yeasts, has one of the highest values (bccresearch.com/market-research/food-and-beverage/the-global-market-for-carotenoids.html).

### Proteins

In some embodiments, the bioproduct is a protein. In some embodiments, the bioproduct is an enzyme. In some aspects, the enzyme is a microbial hydrolytic enzyme. In some aspects, the enzyme is a proteolytic enzyme. In some aspects, the enzyme is applicable to the food, textile, pharmaceutical, or waste management industry (de Souza P M, et al. A biotechnology perspective of fungal proteases. Braz J Microbiol. 2015; 46(2):337-346).

The present description is made with reference to the accompanying drawings and Examples, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they disclose essential matter.

### EXAMPLES

### Example 1: Enzymatic Lysis and Extraction of Lipids from R. toruloides Using plMAN5c

Purified recombinant β-1,3-glucomannanase from Pichia pastoris (plMAN5c) was produced following procedures outlined in Yang et al. 2011 “Purification and Characterization of a β-1,3-Glucomannanase Expressed in Pichia Pastoris.” Enzyme and Microbial Technology 49 (2): 223-28. In brief, methanol-induced cultures (1.5 L of BMMY) were harvested by centrifugation to recover enzyme secreted into the culture supernatant. The protein was precipitated by addition of 800 g ammonium sulfate (reaching 80% ammonium sulfate saturation) and allowed to equilibrate overnight at 4° C. The precipitate was collected by centrifugation at 17000×g for 60 min at 4° C., then resuspended in 1 mM potassium phosphate buffer pH 6.0 containing cOmplete Protease Inhibitor cocktail (Roche CO-RO). This was followed by buffer exchange by dialysis (10 kDa MWCO, 22 mm, ThermoFisher 68100), spin-concentration (Amicon #UFC901008 10 kDa MWCO), and further fractionation by anion-exchange chromatography using a DEAE-Sepharose substrate (Sigma-Aldrich DFF100) at 10 ml scale (Marvelgent Biosciences 11-0257-050), with the active component in the void fraction. Enzyme was stored at 4° C. and used fresh or diluted to 30% glycerol and frozen at −20° C. The enzyme was quantified on an SDS-PAGE gel against a BSA ladder.

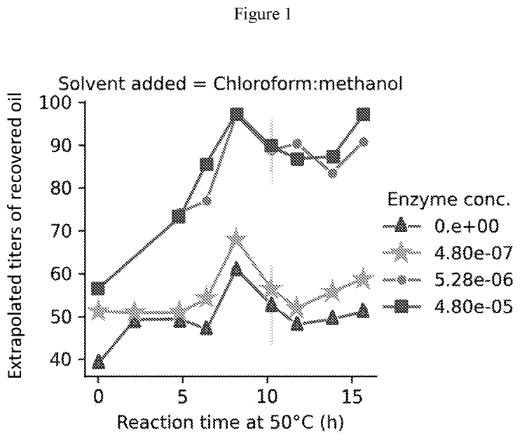

plMAN5c was added at 3 different concentrations, 4.80e-5, 5.28e-6, and 4.80e-7 grams enzyme/gram dry cell weight, in different flasks containing fresh R. toruloides cell culture. These conditions were compared to a control condition without enzyme added (0.e+00). The pH was adjusted to the range of pH 4-4.5 and the flasks were shaken at 50° C. for 16 hours. Every 2 hours, two samples of 10 mL each of the reactions were transferred to 50 mL conical tubes. 15 mL of chloroform:methanol 2:1 or 15 mL of heptane (data not shown) were added to the samples and extracted with light shaking for 3 hours (chloroform:methanol) or 16 hours (heptane). The organic solvent layer was transferred to a new tube, evaporated, and the resulting oil was weighed. Briefly, from a volume of solvent+oil mixture, a small amount was sampled and weight. The solvent was then evaporated, and the residual oil measured. With the relative ratio of oil to solvent in the sample thereby calculated, this fraction was used to estimate the total amount of oil in the total solvent+oil mixture. The “extrapolated” values are shown in FIG. 1. As shown in FIG. 1, there was significantly greater recovered oil titer for both the 5.28e-6 and 4.80e-5 enzyme concentration conditions than for the no enzyme condition at all measured time points.

### Example 2: Purpureocillium lilacinum Secretes Lytic Enzymes which can Solubilize R. toruloides Biomass

Filamentous fungal isolates were obtained from the American Type Culture Collection: Purpureocillium lilacinum ATCC 36010; T. reesei=ATCC 56765. The lyophilized spore suspensions were rehydrated by the addition of 0.5 ml sterile water, then diluted into 5 ml total volume and 4 μl was streaked onto agar media. Spore suspensions were prepared by scraping from PDA plates (>7 days incubation) with 5 ml sterile water, then mixed to 20% final concentration of glycerol and stored at −80° C. Working stocks were stored in water at 4° C. for up to 6 months. All cultures were incubated at 30° C.

R. toruloides CBS 6016 biomass was obtained from high-density fermentation in lipogenic media or from YPD culture in shake-flask, incubated for 3 days at 30° C. Enrichment Medium, based on the formulation of Murao et al., contained 50 g/L wet cell weight R. toruloides biomass obtained from culture in rich or lipogenic medium (EM-YPD or EM-MMG respectively), 5 g/L KH₂PO₄, and 0.5 g/L MgSO₄ heptahydrate. The initial pH was ˜7. Supplemented Enrichment Medium (SEM), pH˜5-6, contained 10 g/L wet cell mass, 10 g/L glucose, 10 g/L meat extract, 5 g/L KH₂PO₄, and 0.5 g/L MgSO₄ heptahydrate. Agar media also contained 15 g/L agar as a solidifying agent. Shake-flask cultures were performed in 100 ml volume in 500 ml shake-flasks, 120 rpm, 30° C., 3 days. All media were autoclave sterilized for 30 minutes.

Halo assays were performed to demonstrate the lytic activity of fungus cultivated on agar containing RT biomass as the major carbon source. This assumes the agar is metabolically inert and that trace media in the wet cell biomass is negligible. In order to assimilate the carbon, the fungal culture must secrete lytic enzymes into the surrounding media, which can solubilize the biomass and cause a visually discernable “clear zone” or halo to form around the fungal colony. This has been demonstrated for P. lilacinum culture on R. glutinis biomass (Arai, M., & Murao, S. (1978). Red yeast cell lysis by red yeast cell wall lytic enzyme and protease. Agricultural and Biological Chemistry, 42(8), 1461-1467), but not in R. toruloides. EM agar were prepared from R. toruloides biomass cultured at both high and low C:N ratio.

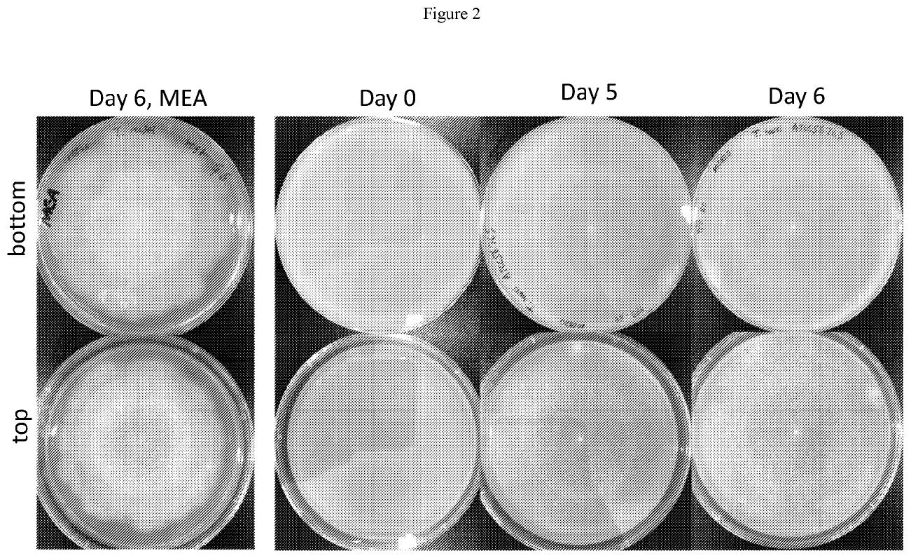

Halo assays were performed by scraping some spores from fungal mycelium and stabbing the spores into the center of EM agar and incubating at 30° C. The wet cell weight was tested from both MMG and YPD to represent R. toruloides biomass obtained from both high and low C:N ratio. The halo assay results with T. reesei demonstrated a sparse mycelial growth, likely due to residual media in the wet cell mass. No discernable halo formed in media derived from either the MM- or YPD-cultured R. toruloides biomass (FIG. 2).

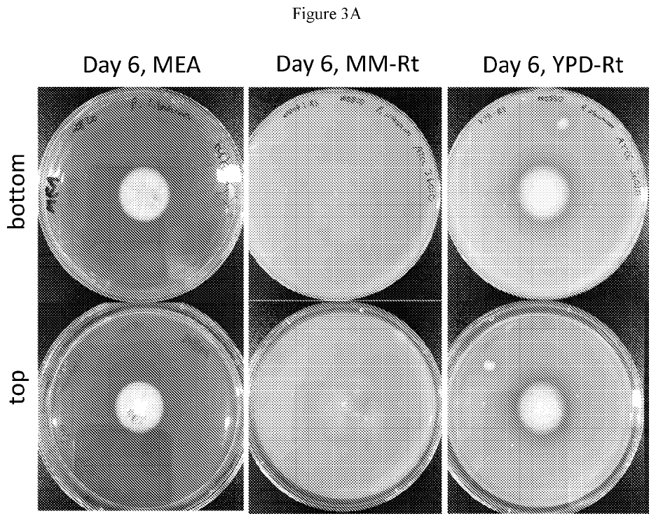

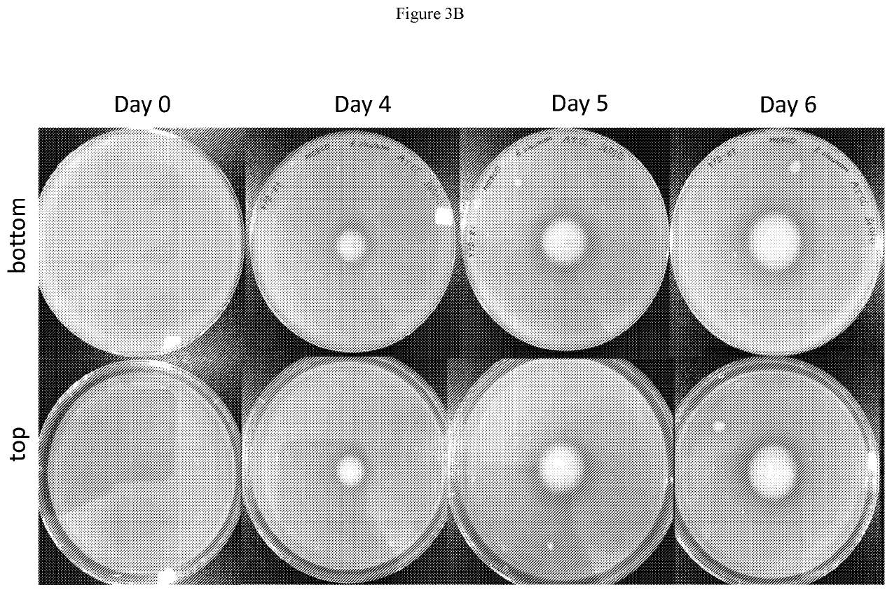

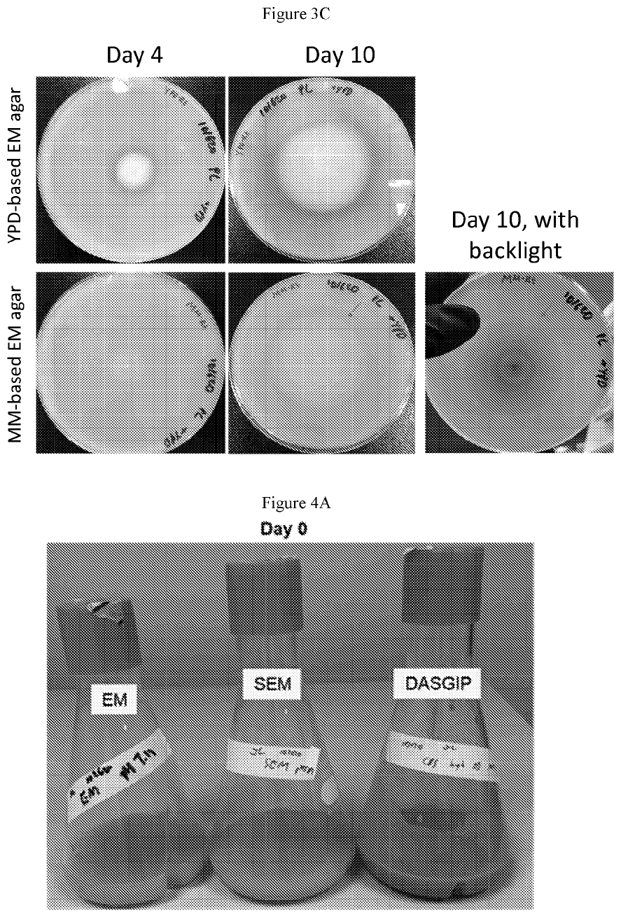

Conversely, halo assay results using PL demonstrated robust formation of lytic zones along the expanding edge of the P. lilacinum mycelium (FIG. 3A). The growth observed in these cultures was comparable to that of P. lilacinum on MEA, a rich medium, indicating robust absorption of nutrients from the R. toruloides biomass. The growth difference on the MMG-based medium reflects the lower available N in that biomass.

A time course analysis of the halo formation by P. lilacinum on YPD-based EM agar is shown in FIG. 3B. The dark clear zone expands with the colony perimeter, showing clear solubilization of the R. toruloides biomass over time.

To confirm these preliminary halo assays, follow-up experiments were conducted on MM-based EM agar. The enrichment of the medium with YPD overlay, combined with longer incubation time, demonstrated definitively that P. lilacinum also hydrolyzes the oleaginous R. toruloides biomass (FIG. 3C).

### Example 3: Live P. lilacinum Mediated Extraction of Lipids from R. toruloides Biomass

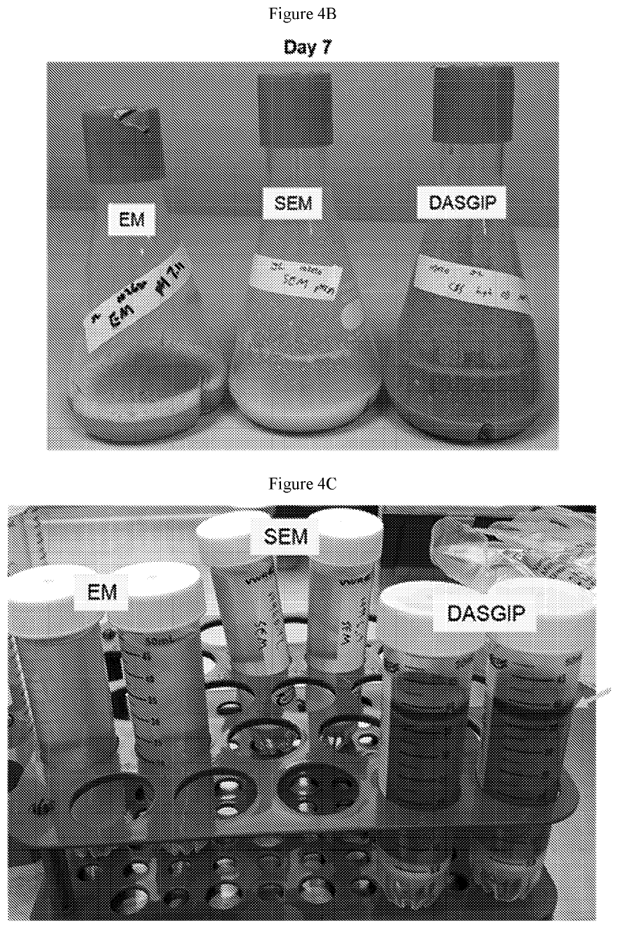

Several 100-ml P. lilacinum shake-flask cultures were prepared to obtain culture broth for assays. One tested condition featured P. lilacinum cultured directly in a preculture of oleaginous yeast grown to suturing density that further had been sterilized for one hour in the autoclave following completion of fermentation. Cultures with robust growth of the P. lilacinum (SEM and DASGIP) demonstrated caking of oily biomass on the side walls of the flasks, and the DASGIP flask also displayed red oil streaks (FIGS. 4A and 4B).

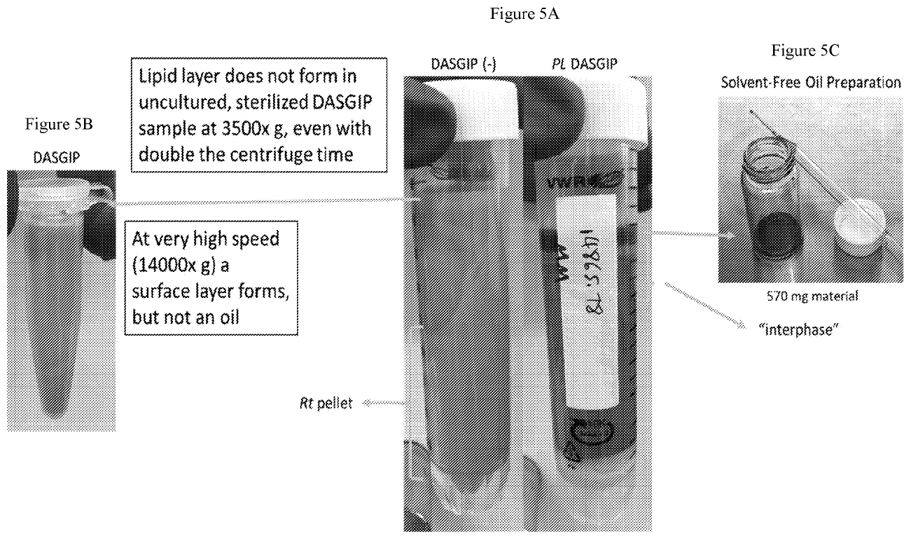

To confirm the necessity of P. lilacinum to induce the free oil formation, uncultured sterile DASGIP broth was centrifuged in similar conditions (3500×g, 20 min) (FIG. 4C). Even with double the centrifuge time, or quadruple the g-force, no free oil could be formed without the lytic effect of P. lilacinum, although a layer of floating cell debris was seen at the surface of the supernatant (FIG. 4B). This observed buoyant layer is consistent with segregation of free lipid bodies from whole cells in a R. toruloides sample degraded by a harsh one-hour autoclave pretreatment and could suggest that R. toruloides lipid bodies pose a significant barrier to free oil formation due to their stabilizing surface proteins. The oil phase was directly harvested from the P. lilacinum samples by pipetting, demonstrating the first reported instance of R. toruloides oil prepared without the use of organic solvents (FIG. 5C). The resulting sample was entirely solvent-free.

### Example 4: Compositional Analyses of Oils from P. lilacinum Extractions of R. toruloides Biomass

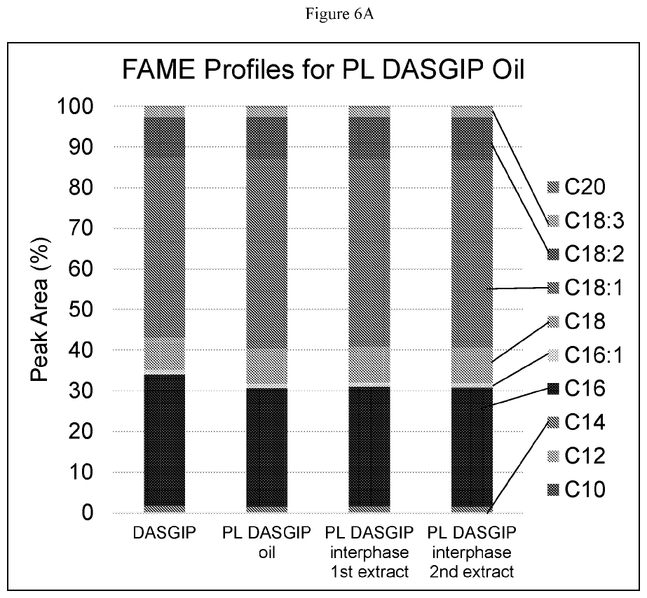

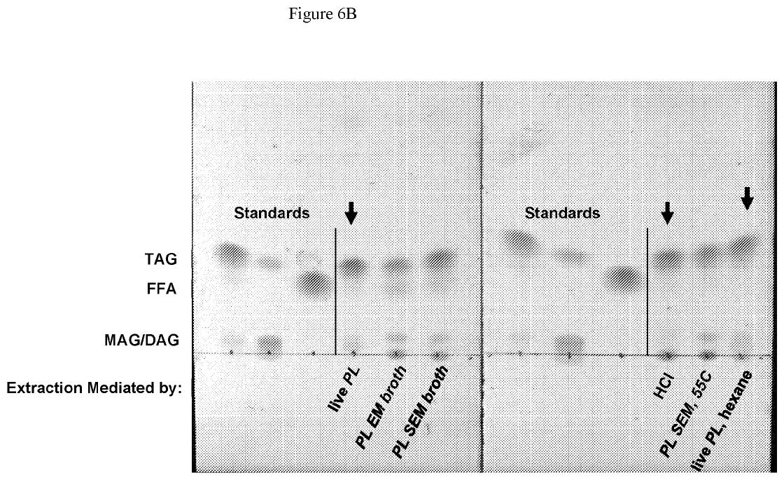

The P. lilacinum extraction sample was characterized by GC-FID and TLC to quantify the FAME profile and approximate the free fatty acid (FFA) content respectively. FAME analyses showed no difference between the gravity-separated or solvent-extracted oil. However, a slight difference was observed in the oil samples after contact with P. lilacinum, namely increased C18:2 content and decreased C16:0 content (FIG. 6A). In terms of FFA, P. lilacinum extracted oil is similar to the benchmark HCl-chloroform-methanol extracted oil (FIG. 6B).

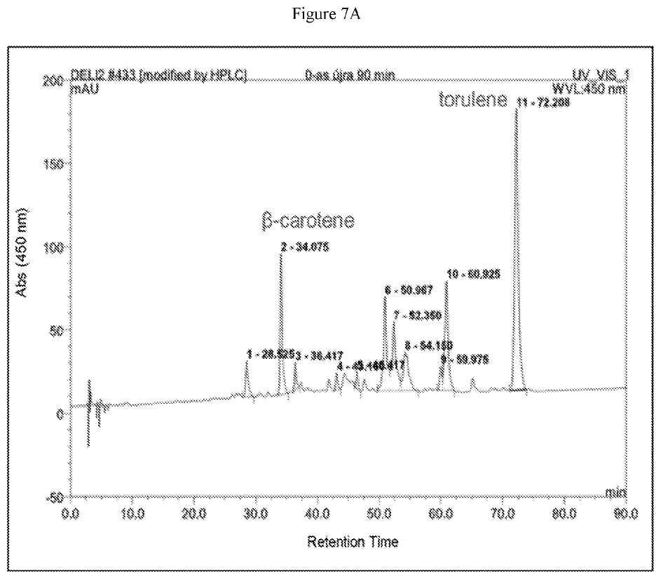

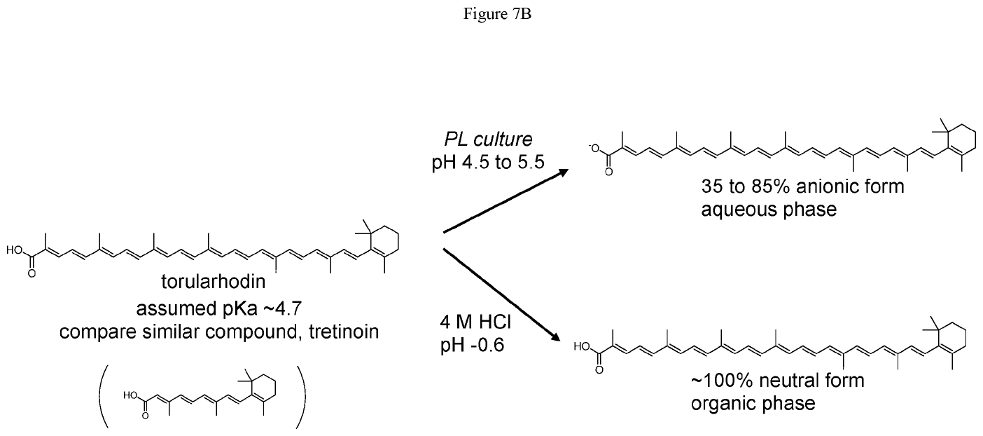

Samples of the solvent-free and solvent-extracted oils were sent to a third party—Carotenature, for analysis of carotenoid content by LC-DAD (FIG. 7A). R. toruloides is known to accumulate three main carotenoids, β-carotene, torulene, and torularhodin. Interestingly, the β-carotene and torulene content was 2-4× times higher than that of other acid-treated oil extracts (36% torulene, 11% β-carotene), but torularhodin was notably absent (FIG. 7A, Table 3 below). One explanation is that torularhodin, a carboxylic acid, is partially or majorly ionized under the pH conditions of P. lilacinum culture, whereas after acidic treatment, the majority of torularhodin should be in the protonated neutral form and easily partitions into the hydrophobic solvent during extraction (FIG. 7B).

TABLE 3
HPLC peak identification

| Peak | Ret. Time | | | Area | Rel. | |
|---|---|---|---|---|---|---|
| No. | (min) | Peak Name | λₘₐₓ (nm) | mAU*min | Area (%) | Type |
| 1 | 28.53 | (13Z)-β-Carotene | 443, 469 | 10.770 | 3.47 | BMB |
| 2 | 34.08 | β-Carotene | 451, 477 | 34.796 | 11.20 | BMB |
| 3 | 36.42 | (9Z)-β-Carotene | 446, 471 | 5.851 | 1.88 | BMB |
| 4 | 43.15 | unidentified (ui) | 434, 456, 484 | 3.528 | 1.14 | BMB |
| 5 | 46.42 | ui | Not detected | 3.873 | 1.25 | BMB |
| 6 | 50.97 | ui Z-isomer | 381, 480 | 34.250 | 11.03 | BM* |
| 7 | 52.35 | ui | 452, 479, 511 | 30.971 | 9.97 | M* |
| 8 | 54.15 | ui. | 449, 473, 503 | 24.712 | 7.96 | MB* |
| 9 | 59.98 | ui | 452, 477, 508 | 5.799 | 1.87 | BM* |
| 10 | 60.93 | ui. | 457, 482, 514 | 42.164 | 13.58 | MB* |
| 101 | 72.21 | Torulene | 461, 486, 519 | 113.867 | 36.66 | BMB |
| Total: | | | | 310.583 | 100.00 | |

### Example 5: Optimization of Live P. lilacinum Mediated R. toruloides Oil Extraction

The shake-flask live P. lilacinum mediated oil extraction was repeated several times successfully with different batches of DASGIP broth containing different levels of residual nutrients. It was observed that in P. lilacinum mediated mycelial extractions, the caked biomass ring that forms at wall of the shake-flask sometimes show liquefying R. toruloides. One possibility is that the active proteins are secreted from the P. lilacinum at these zones.

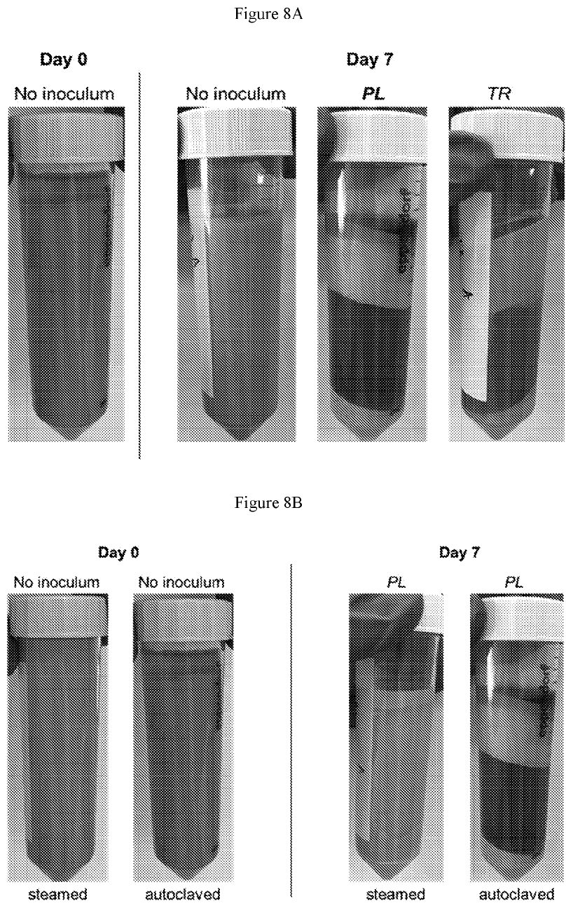

Unexpectedly, T. reesei demonstrated a partial activity to release the oil from DASGIP suspension. T. reesei was shown to grow on the halo assay media and would theoretically hydrolyze β-(1,3)-mannan elements in the proposed model of the R. toruloides cell wall. When incubated with DASGIP suspension over 7 days culture, the T. reesei culture formed a buoyant, lipid-body enriched phase upon centrifugation (FIG. 8A). The formation of free oil, though, is unique to P. lilacinum treatment. These observations could be explained by the interpretation that both filamentous fungi can lyse the cell walls and release the buoyant lipid body fraction, but only P. lilacinum can further disrupt the lipid bodies and allow the lipids to coalesce into a free oil. Another explanation would be that T. reesei can disrupt the lipid bodies as well, but rapidly consumes the lipid, leaving no oil.

Methods of inactivating the R. toruloides biomass (physical pretreatment) were also tested (FIG. 8B). DASGIP suspension sterilized by steaming for one hour in the water bath did not form a free oil when used to cultivate P. lilacinum, whereas the physical pretreatment of autoclaving does.

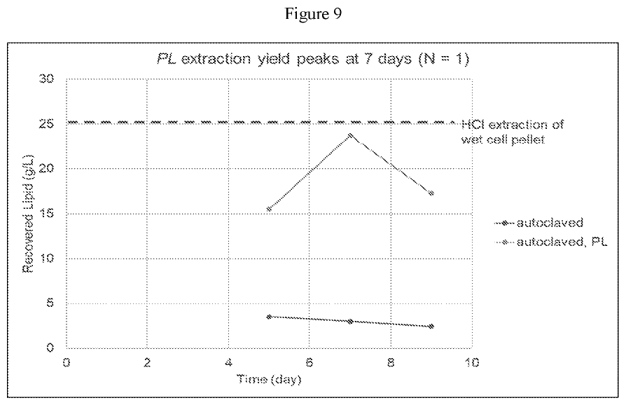

A preliminary experiment was made to optimize the duration of P. lilacinum extraction in the direct culture approach (FIG. 9). Two 100-ml shake-flasks were set up and one was inoculated with P. lilacinum. Aliquots of 10 ml were collected at days 5, 7, and 9 and extracted directly with hexanes. The results from a single shake-flask showed a peak in oil formation at 7 days.

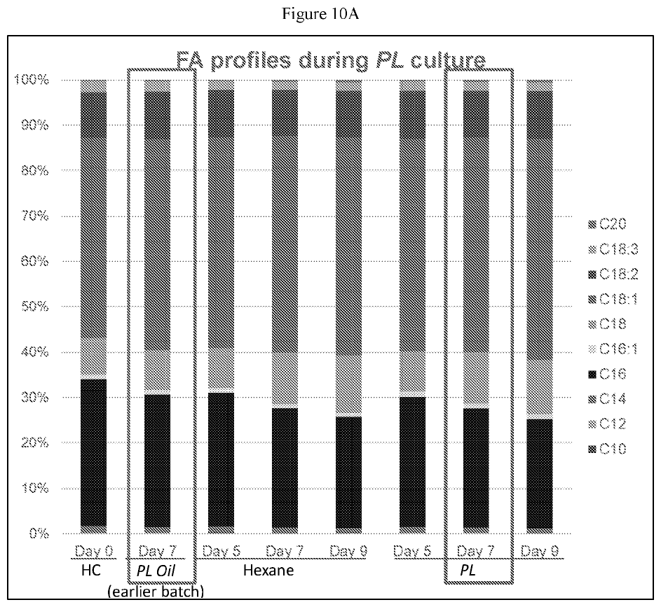

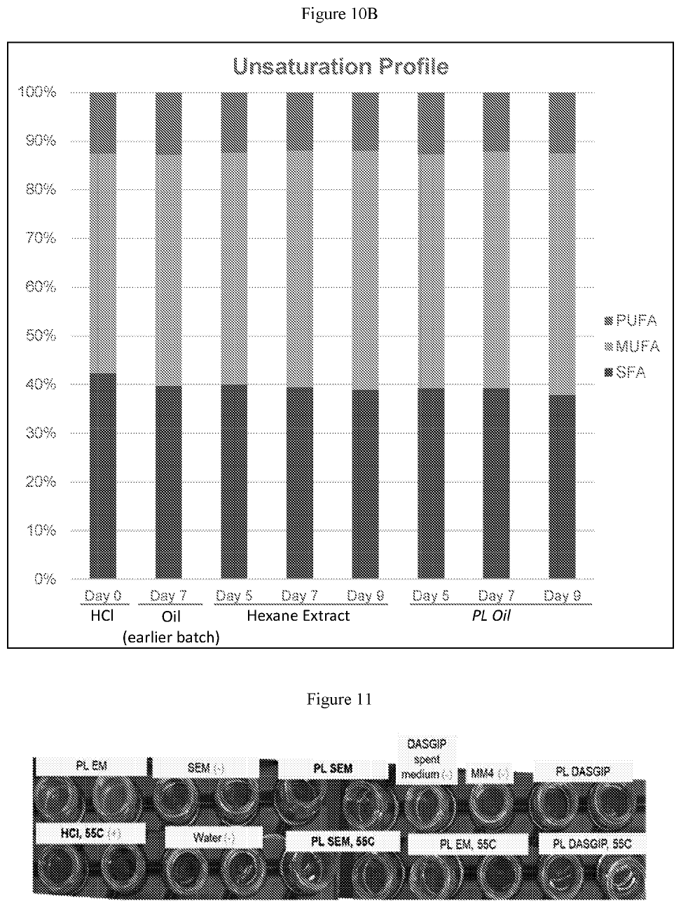

FAME analysis demonstrated a trend in the fatty acid profiles over the course of P. lilacinum extraction, with C16:0 converting to C18:0 and C18:1. When compared to earlier P. lilacinum extracted oils, little variance (˜3%) was detected in the day 7 samples (FIG. 10A). Unsaturation profile showed at least 30% w/w saturated fatty acids (SFA), at least 30% w/w unsaturated fatty acids (“mono-unsaturated” MUFA), and less than 30% w/w total polyunsaturated fatty acids (PUFA) (FIG. 10B).

### Example 6: Enzymatic Lysis and Extraction of Lipids from R. toruloides Wet Biomass Using Enzyme Cocktails

Larger-scale assays using wet cell mass were conducted based on the assumption that a typical endpoint oleaginous yeast culture contains ˜400 mg dried cell weight per 3.64 ml. Wet cell pellets were prepared by the following method: culture was suspended by pipetting, then split into 4 ml aliquot in 50 ml centrifuge tubes, with five extra replicates to measure the equivalent dried cell mass. Aliquots were pelleted at 4000×g, the supernatant decanted, then the tubes tapped dry on paper towels. The five extra aliquots were dried at 55° C. in the vacuum oven and massed. The starting material contained the equivalent of approximately 396 mg (±1.27%) dried cell weight per tube.

The enzyme mixes, comprising secreted enzymes produced by P. lilacinum in different media, were prepared by shake-flask fermentation of P. lilacinum in EM, SEM, Medium F, and sterilized oleaginous yeast suspension. For the treated conditions, incubation with enzyme cocktails was followed by solvent extraction. For the controls, extraction was carried out with acid lysis plus solvent extraction. After extraction, the level of oil residue and pigmentation was notable in the acid-treated samples and the PL SEM broth treated samples (FIG. 11), which could be indicative of higher lipid recovery.

The P. lilacinum SEM samples had the highest average yields (data not shown). These results suggest that P. lilacinum in SEM has the highest activity at 7 days, likely because it is the medium with the most nitrogen content. Thus there is an opportunity to optimize activity of the P. lilacinum broth by harvesting at earlier time points, or by culturing in even richer media on substrates like amylopectin or chitin to facilitate denser growth and robust enzyme secretion.

Combined, these data demonstrate the potential of P. lilacinum to replace both the acid hydrolysis and solvent extraction stages of current lysis-bioproduct extraction methods. P. lilacinum cultured this way could be separated by simple centrifugation to yield a free-flowing oil phase without the need for chemical solvents.

### Numbered Embodiments of the Invention

Notwithstanding the appended claims, the disclosure sets forth the following numbered embodiments:
1. A method for isolating a bioproduct from a yeast comprising:
treating yeast cells with a β-1,3-glucomannanase, wherein the β-1,3-glucomannanase is in an amount of less than 1.0e-4 g enzyme protein/g dry cell weight, thereby producing an enzymatically lysed sample;
separating the lipid phase from the aqueous phase of the enzymatically lysed sample via solvent or non-solvent extraction, thereby producing a separated sample; and
isolating a bioproduct from the separated sample.
2. The method of embodiment 1, wherein the β-1,3-glucomannanase is in an amount of between 1.0e-6 and 5.0e-5 g enzyme protein/g dry cell weight.
3. The method of embodiment 1 or 2, wherein the β-1,3-glucomannanase is an isolated and purified recombinant protein.
4. The method of embodiment 3, wherein the β-1,3-glucomannanase is expressed and purified from Pichia pastoris.
5. The method of embodiment 1 or 2, wherein the β-1,3-glucomannanase is produced and/or secreted by a cellulolytic fungi.
6. The method of any one of embodiments 1-5, wherein the method comprises a physical pre-treatment of the yeast cells prior to treating with β-1,3-glucomannanase.
7. The method of embodiment 5, wherein the cellulolytic fungi is a species of Trichoderma, Humicola, Purpureocillium, Penicillium, Phanerochaete, or Pycnoporus.
8. The method of embodiment 7, wherein the cellulolytic fungi is Purpureocillium lilacinum, Penicillium pinophilum, Penicilium brasilinum, Trichoderma reesei, or Humicola insolens.
9. The method of any one of embodiments 1-3, or 5-8, wherein the β-1,3-glucomannanase is produced or secreted by a genetically modified Purpureocillium lilacinum.
10. The method of any one of embodiments 1-3, or 5-8, wherein the β-1,3-glucomannanase is produced or secreted by a genetically modified Trichoderma reesei.
11. The method of any one of embodiments 1-10, wherein the β-1,3-glucomannanase is a part of an enzyme cocktail.
12. The method of any one of embodiments 5-10, wherein the β-1,3-glucomannanase is comprised within a blended enzyme extract from two or more microorganisms.
13. The method of any one of embodiments 1-12, wherein the treating yeast cells with a 0-1,3-glucomannanase occurs at between 20° C. and 55° C.
14. The method of embodiment 13, wherein the treating yeast cells with a β-1,3-glucomannanase occurs at 50° C.
15. The method of any one of embodiments 1-14, wherein the yeast cells are treated with the β-1,3-glucomannanase for between 5 and 15 hours.
16. The method of embodiment 15, wherein the yeast cells are treated with the β-1,3-glucomannanase for approximately 10 hours.
17. The method of any one of embodiments 1-16, wherein the treating yeast cells with a 0-1,3-glucomannanase occurs at a pH of between 4 and 4.5.
18. The method of any one of embodiments 1-17, wherein the separation is performed via solvent extraction.
19. The method of embodiment 18, wherein the solvent is hexane, heptane, or chloroform and methanol.
20. The method of embodiment 18, wherein the solvent is hexane.
21. The method of embodiment 18, wherein the solvent is heptane.
22. The method of embodiment 18, wherein the solvent is chloroform and methanol.
23. The method of embodiment 22, wherein the chloroform:methanol ratio is 2:1.
24. The method of embodiment 18, wherein the solvent is not ethyl acetate.
25. The method of any one of embodiments 18-24, wherein solvent extraction is performed at between 30° C. and 55° C.
26. The method of any one of embodiments 18-25, wherein solvent extraction is carried out for about 7-10 hours.
27. The method of any one of embodiments 18-26, wherein solvent extraction is carried out for about 10-16 hours.
28. The method of any one of embodiments 18-27, wherein a phospholipid solvent is added during extraction.
29. The method of any one of embodiments 18-28, wherein ethanol, methanol, or acetone is added during extraction.
30. The method of any one of embodiments 18-29, wherein an additional enzyme is added prior to or during extraction.
31. The method of any one of embodiments 18-30, wherein the yeast cells are treated with the β-1,3-glucomannanase and the solvent at the same time.
32. The method of any one of embodiments 1-32, wherein the separation is carried out via non-solvent extraction.
33. The method of embodiment 32, wherein the separation is carried out via gravimetric separation.
34. The method of any one of embodiments 1-33, further comprising a mechanical treatment between the lysis and extraction.
35. The method of embodiment 34, wherein the mechanical treatment is at least one of bead milling, ultrasonication, high-pressure homogenization, shearing, and microwave irradiation.
36. The method of any one of embodiments 1-35, further comprising an acid lysis.
37. The method of any one of embodiments 1-36, wherein the yeast is an oleaginous yeast.
38. The method of embodiment 37, wherein the yeast is a species from the Rhodotorula, Rhodosporidium, or Sporobolomyces genus.
39. The method of embodiment 38, wherein the yeast is Rhodosporidium toruloides, Rhodotorula glutinis, Rhodosporidium diobovatum, Rhodosporidium kratochvilovae, Rhodotorula graminis, Rhodotorula babjevae, and Rhodotorula taiwanensis.
40. The method of any one of embodiments 1-39, wherein the bioproduct is a lipid, carotenoid, enzyme, saccharide, or combination thereof.
41. The method of embodiment 40, wherein the bioproduct is a lipid.
42. A method for enzymatic lysis of microorganisms having recalcitrant cell walls comprising:
inactivating a biomass of microorganisms having recalcitrant cell walls;
inoculating the inactive biomass with live cellulolytic fungi and/or an organism engineered to express at least one cellulolytic enzyme; and
incubating the live cellulolytic fungi and/or an organism engineered to express at least one cellulolytic enzyme for at least 5 hours to generate a lysed biomass.
43. The method of embodiment 42, wherein the inactive biomass to live cell ratio is between 10,000:1 and 1:1 dry cell w/w.
44. The method of embodiment 42, wherein the inactive biomass to live cell ratio is between 10,000:1 and 10:1 dry cell w/w.
45. The method of embodiment 42, wherein the inactive biomass to live cell ratio is between 10,000:1 and 100:1 dry cell w/w.
46. The method of any one of embodiments 42-45, wherein the incubating is for at least 10 hours.
47. The method of any one of embodiments 42-46, wherein the incubating occurs at between 20° C. and 55° C.
48. The method of any one of embodiments 42-47, wherein the cellulolytic fungi is a species of Trichoderma, Humicola, Penicillium, Purpureocillium, Phanerochaete, or Pycnoporus.
49. The method of any one of embodiments 42-48, wherein the organism engineered to express at least one cellulolytic enzyme is Pichia pastoris, Purpureocillium lilacinum, or Trichoderma reesei.
50. The method of any one of embodiments 42-49, further comprising isolating a bioproduct from the lysed biomass.
51. The method of embodiment 50, wherein the bioproduct is a lipid, carotenoid, enzyme, saccharide, or combination thereof.
52. The method of embodiment 50, wherein the bioproduct is lipids, and wherein the lipids are isolated by gravimetric separation.
53. The method of any one of embodiments 42-52, further comprising a mechanical pre-treatment.
54. The method of embodiment 53, wherein the mechanical pre-treatment is pressure, ultrasonication, or microwave irradiation.
55. A composition comprising:
two or more enzymes, wherein the two or more enzymes comprise an isolated and purified 3-1,3-glucomannanase and at least one of a cellulase and a protease; and
an inactive cell biomass,
wherein the composition has a total grams enzyme to dry cell weight ratio between 1:10,000 and 1:1,000,000.
56. The composition of embodiment 55, wherein the inactive cell biomass comprises one or more species of Rhodotorula, Rhodosporidium, or Sporobolomyces.
57. The composition of embodiment 55 or 56, wherein the inactive cell biomass comprises Rhodosporidium toruloides.
58. The composition of any one of embodiments 55-57, wherein the enzyme to dry cell weight ratio is between 1:10,000 and 1:100,000.
59. A composition comprising a live cellulolytic fungus and an inactive yeast, wherein the cellulolytic fungus is a species selected from Trichoderma, Humicola, Penicillium, Purpureocillium, Phanerochaete, and Pycnoporus, and wherein the inactive yeast is a species selected from Rhodotorula, Rhodosporidium, or Sporobolomyces, and wherein the inactive yeast to live cellulolytic fungus ratio is between 10,000:1 and 1:1 dry cell w/w.
60. The composition of embodiment 59, wherein the cellulolytic fungus produces β-1,3-glucomannanase.
61. The composition of embodiment 59, wherein the cellulolytic fungus has been genetically engineered to produce 1,3-glucomannanase.
62. The composition of any one of embodiments 59-61, wherein the cellulolytic fungus is Purpureocillium lilacinum and the inactive yeast is Rhodosporidium toruloides.
63. The composition of any one of embodiments 59-62, wherein the inactive yeast has been genetically modified to produce a bioproduct.
64. The composition of any one of embodiments 59-63, wherein the inactive yeast to live cellulolytic fungus ratio is between 1000:1 and 10:1 dry cell w/w.
65. The composition of any one of embodiments 59-63, wherein the inactive yeast to live cellulolytic fungus ratio is between 1000:1 and 100:1 dry cell w/w.
66. A bioproduct isolated from the composition of any one of embodiments 59-65, wherein the isolated bioproduct does not comprise a detectable amount of a solvent.
67. A microbial oil produced by an oleaginous yeast, wherein the oil comprises less than 10 ppm of a solvent, and at least one pigment selected from the group consisting of carotene, torulene and torulorhodin.
68. The microbial oil of embodiment 67, wherein the oil comprises less than 8 ppm of a solvent.
69. The microbial oil of embodiment 67, wherein the oil comprises less than 6 ppm of a solvent.
70. The microbial oil of embodiment 67, wherein the oil comprises less than 4 ppm of a solvent.
71. The microbial oil of embodiment 67, wherein the oil comprises less than 2 ppm of a solvent.
72. The microbial oil of embodiment 67, wherein the oil does not comprise a detectable amount of a solvent.
73. The microbial oil of any one of embodiments 67-77, wherein the solvent is heptane, hexane, ethyl acetate, ethanol, chloroform, and/or methanol.
74. The microbial oil of any one of embodiments 67-73, wherein the oil comprises a fatty acid profile comprising:
at least 30% w/w saturated fatty acids;
at least 30% w/w unsaturated fatty acids; and
less than 30% w/w total polyunsaturated fatty acids.
75. The microbial oil of any one of embodiments 67-74, wherein the oil comprises (3-carotene and torulene.
76. The microbial oil of any one of embodiments 67-75, wherein the oil comprises at least 10 ppm, at least 50 ppm, or at least 100 ppm torulene
77. The microbial oil of any one of embodiments 67-76, wherein the fatty acid profile comprises:
greater than 40% w/w saturated fatty acids;
greater than 40% w/w mono-unsaturated fatty acids; and
less than 20% w/w polyunsaturated fatty acids.
78. The microbial oil of any one of embodiments 67-77, wherein the oil comprises the following amounts of fatty acids relative to the total fatty acids:
between about 7.0% and 35% stearic acid;
between about 10% and 50% oleic acid; and
between about 8% and 20% linoleic acid.
79. An autolytic yeast that produces a bioproduct, wherein the yeast comprises a gene encoding a cellulolytic enzyme, and wherein expression of the gene is under the control of an inducible promoter.
80. The autolytic yeast of embodiment 79, wherein the yeast further comprises one or more targeted modifications to the secretory and trafficking pathways.
81. The autolytic yeast of embodiments 79 or 80, wherein the gene is MAN5C from Purpureocillium lilacinum.
82. The autolytic yeast of any one of embodiments 79-81, wherein the cellulolytic enzyme is β-1,3-glucomannase.
83. The autolytic yeast of any one of embodiments 79-82, wherein the cellulolytic enzyme is targeted for extracellular secretion.
84. The autolytic yeast of any one of embodiments 79-83, wherein the cellulolytic enzyme is targeted to an intracellular compartment.
85. The autolytic yeast of any one of embodiments 79-84, wherein the yeast is Rhodosporidium toruloides.
86. An autolytic method of producing a bioproduct from an industrious yeast comprising: genetically engineering an industrious yeast to express and/or secrete a cellulolytic enzyme, wherein the industrious yeast produces a bioproduct, and wherein the expression of the cellulolytic enzyme is under the control of an inducible promoter;
growing the yeast to produce the bioproduct;
inducing expression of the cellulolytic enzyme to autolyse the yeast; and
extracting, isolating, and/or purifying the bioproduct.
87. The method of embodiment 86, wherein the yeast is Rhodosporidium toruloides.
88. The method of embodiment 86 or 87, wherein the genetically engineering comprising inserting the MAN5C gene from Purpureocillium lilacinum.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world. The following international PCT applications are incorporated herein by reference their entireties: International Patent Application Nos. PCT/US2021/59147, and PCT/US2021/59122, and International Patent Publication Nos. WO2021/163194, and WO2021/154863.

1. A method for isolating a bioproduct from a yeast comprising:
treating yeast cells with a β-1,3-glucomannanase, wherein the β-1,3-glucomannanase is in an amount of less than 1.0e-4 g enzyme protein/g dry cell weight, thereby producing an enzymatically lysed sample;
separating the lipid phase from the aqueous phase of the enzymatically lysed sample via solvent or non-solvent extraction, thereby producing a separated sample; and
isolating a bioproduct from the separated sample.

2. The method of claim 1, wherein the β-1,3-glucomannanase is in an amount of between 1.0e-6 and 5.0e-5 g enzyme protein/g dry cell weight.

3. The method of claim 1, wherein the β-1,3-glucomannanase is an isolated and purified recombinant protein.

4. The method of claim 3, wherein the β-1,3-glucomannanase is expressed and purified from Pichia pastoris.

5. The method of claim 1, wherein the β-1,3-glucomannanase is produced and/or secreted by a cellulolytic fungi.

6. The method of claim 1, wherein the method comprises a physical pre-treatment of the yeast cells prior to treating with β-1,3-glucomannanase.

7. The method of claim 5, wherein the cellulolytic fungi is a species of Trichoderma, Humicola, Purpureocillium, Penicillium, Phanerochaete, or Pycnoporus.

8. The method of claim 7, wherein the cellulolytic fungi is Purpureocillium lilacinum, Penicillium pinophilum, Penicilium brasilinum, Trichoderma reesei, or Humicola insolens.

9. The method of claim 1, or 5, wherein the β-1,3-glucomannanase is produced or secreted by a genetically modified Purpureocillium lilacinum.

10. The method of claim 1, or 5, wherein the β-1,3-glucomannanase is produced or secreted by a genetically modified Trichoderma reesei.

11. The method of claim 1, wherein the β-1,3-glucomannanase is a part of an enzyme cocktail.

12. The method of claim 5, wherein the β-1,3-glucomannanase is comprised within a blended enzyme extract from two or more microorganisms.

13. The method of claim 1, wherein the treating yeast cells with a β-1,3-glucomannanase occurs at between 20° C. and 55° C.

14. The method of claim 13, wherein the treating yeast cells with a β-1,3-glucomannanase occurs at 50° C.

15. The method of claim 1, wherein the yeast cells are treated with the β-1,3-glucomannanase for between 5 and 15 hours.

16. The method of claim 15, wherein the yeast cells are treated with the β-1,3-glucomannanase for approximately 10 hours.

17. The method of claim 1, wherein the treating yeast cells with a β-1,3-glucomannanase occurs at a pH of between 4 and 4.5.

18. The method of claim 1, wherein the separation is performed via solvent extraction.

19. The method of claim 18, wherein the solvent is hexane, heptane, or chloroform and methanol.

20. The method of claim 18, wherein the solvent is hexane.

21. The method of claim 18, wherein the solvent is heptane.

22. The method of claim 18, wherein the solvent is chloroform and methanol.

23. The method of claim 22, wherein the chloroform:methanol ratio is 2:1.

24. The method of claim 18, wherein the solvent is not ethyl acetate.

25. The method of claim 18, wherein solvent extraction is performed at between 30° C. and 55° C.

26. The method of claim 18, wherein solvent extraction is carried out for about 7-10 hours.

27. The method of claim 18, wherein solvent extraction is carried out for about 10-16 hours.

28. The method of claim 18, wherein a phospholipid solvent is added during extraction.

29. The method of claim 18, wherein ethanol, methanol, or acetone is added during extraction.

30. The method of claim 18, wherein an additional enzyme is added prior to or during extraction.

31. The method of claim 18, wherein the yeast cells are treated with the β-1,3-glucomannanase and the solvent at the same time.

32. The method of claim 1, wherein the separation is carried out via non-solvent extraction.

33. The method of claim 32, wherein the separation is carried out via gravimetric separation.

34. The method of claim 1, further comprising a mechanical treatment between the lysis and extraction.

35. The method of claim 34, wherein the mechanical treatment is at least one of bead milling, ultrasonication, high-pressure homogenization, shearing, and microwave irradiation.

36. The method of claim 1, further comprising an acid lysis.

37. The method of claim 1, wherein the yeast is an oleaginous yeast.

38. The method of claim 37, wherein the yeast is a species from the Rhodotorula, Rhodosporidium, or Sporobolomyces genus.

39. The method of claim 38, wherein the yeast is Rhodosporidium toruloides, Rhodotorula glutinis, Rhodosporidium diobovatum, Rhodosporidium kratochvilovae, Rhodotorula graminis, Rhodotorula babjevae, and Rhodotorula taiwanensis.

40. The method of claim 1, wherein the bioproduct is a lipid, carotenoid, enzyme, saccharide, or combination thereof.

41. The method of claim 40, wherein the bioproduct is a lipid.

42. A method for enzymatic lysis of microorganisms having recalcitrant cell walls comprising:
inactivating a biomass of microorganisms having recalcitrant cell walls;
inoculating the inactive biomass with live cellulolytic fungi and/or an organism engineered to express at least one cellulolytic enzyme; and
incubating the live cellulolytic fungi and/or an organism engineered to express at least one cellulolytic enzyme for at least 5 hours to generate a lysed biomass.

43. The method of claim 42, wherein the inactive biomass to live cell ratio is between 10,000:1 and 1:1 dry cell w/w.

44. The method of claim 42, wherein the inactive biomass to live cell ratio is between 10,000:1 and 10:1 dry cell w/w.

45. The method of claim 42, wherein the inactive biomass to live cell ratio is between 10,000:1 and 100:1 dry cell w/w.

46. The method of claim 42, wherein the incubating is for at least 10 hours.

47. The method of claim 42, wherein the incubating occurs at between 20° C. and 55° C.

48. The method of claim 42, wherein the cellulolytic fungi is a species of Trichoderma, Humicola, Penicillium, Purpureocillium, Phanerochaete, or Pycnoporus.

49. The method of claim 42, wherein the organism engineered to express at least one cellulolytic enzyme is Pichia pastoris, Purpureocillium lilacinum, or Trichoderma reesei.

50. The method of claim 42, further comprising isolating a bioproduct from the lysed biomass.

51. The method of claim 50, wherein the bioproduct is a lipid, carotenoid, enzyme, saccharide, or combination thereof.

52. The method of claim 50, wherein the bioproduct is lipids, and wherein the lipids are isolated by gravimetric separation.

53. The method of claim 42, further comprising a mechanical pre-treatment.

54. The method of claim 53, wherein the mechanical pre-treatment is pressure, ultrasonication, or microwave irradiation.

55. A composition comprising:
two or more enzymes, wherein the two or more enzymes comprise an isolated and purified β-1,3-glucomannanase and at least one of a cellulase and a protease; and
an inactive cell biomass,
wherein the composition has a total grams enzyme to dry cell weight ratio between 1:10,000 and 1:1,000,000.

56. The composition of claim 55, wherein the inactive cell biomass comprises one or more species of Rhodotorula, Rhodosporidium, or Sporobolomyces.

57. The composition of claim 55 or 56, wherein the inactive cell biomass comprises Rhodosporidium toruloides.

58. The composition of claim 55, wherein the enzyme to dry cell weight ratio is between 1:10,000 and 1:100,000.

59. A composition comprising a live cellulolytic fungus and an inactive yeast, wherein the cellulolytic fungus is a species selected from Trichoderma, Humicola, Penicillium, Purpureocillium, Phanerochaete, and Pycnoporus, and wherein the inactive yeast is a species selected from Rhodotorula, Rhodosporidium, or Sporobolomyces, and wherein the inactive yeast to live cellulolytic fungus ratio is between 10,000:1 and 1:1 dry cell w/w.

60. The composition of claim 59, wherein the cellulolytic fungus produces β-1,3-glucomannanase.

61. The composition of claim 59, wherein the cellulolytic fungus has been genetically engineered to produce 1,3-glucomannanase.

62. The composition of claim 59, wherein the cellulolytic fungus is Purpureocillium lilacinum and the inactive yeast is Rhodosporidium toruloides.

63. The composition of claim 59, wherein the inactive yeast has been genetically modified to produce a bioproduct.

64. The composition of claim 59, wherein the inactive yeast to live cellulolytic fungus ratio is between 1000:1 and 10:1 dry cell w/w.

65. The composition of claim 59, wherein the inactive yeast to live cellulolytic fungus ratio is between 1000:1 and 100:1 dry cell w/w.

66. A bioproduct isolated from the composition of claim 59, wherein the isolated bioproduct does not comprise a detectable amount of a solvent.

67. A microbial oil produced by an oleaginous yeast, wherein the oil comprises less than 10 ppm of a solvent, and at least one pigment selected from the group consisting of carotene, torulene and torulorhodin.

68. The microbial oil of claim 67, wherein the oil comprises less than 8 ppm of a solvent.

69. The microbial oil of claim 67, wherein the oil comprises less than 6 ppm of a solvent.

70. The microbial oil of claim 67, wherein the oil comprises less than 4 ppm of a solvent.

71. The microbial oil of claim 67, wherein the oil comprises less than 2 ppm of a solvent.

72. The microbial oil of claim 67, wherein the oil does not comprise a detectable amount of a solvent.

73. The microbial oil of claim 67, wherein the solvent is heptane, hexane, ethyl acetate, ethanol, chloroform, and/or methanol.

74. The microbial oil of claim 67, wherein the oil comprises a fatty acid profile comprising:
at least 30% w/w saturated fatty acids;
at least 30% w/w unsaturated fatty acids; and
less than 30% w/w total polyunsaturated fatty acids.

75. The microbial oil of claim 67, wherein the oil comprises β-carotene and torulene.

76. The microbial oil of claim 67, wherein the oil comprises at least 10 ppm, at least 50 ppm, or at least 100 ppm torulene

77. The microbial oil of claim 67, wherein the fatty acid profile comprises:
greater than 40% w/w saturated fatty acids;
greater than 40% w/w mono-unsaturated fatty acids; and
less than 20% w/w polyunsaturated fatty acids.

78. The microbial oil of claim 67, wherein the oil comprises the following amounts of fatty acids relative to the total fatty acids:
between about 7.0% and 35% stearic acid;
between about 10% and 50% oleic acid; and
between about 8% and 20% linoleic acid.

79. An autolytic yeast that produces a bioproduct, wherein the yeast comprises a gene encoding a cellulolytic enzyme, and wherein expression of the gene is under the control of an inducible promoter.

80. The autolytic yeast of claim 79, wherein the yeast further comprises one or more targeted modifications to the secretory and trafficking pathways.

81. The autolytic yeast of claim 79, wherein the gene is MAN5C from Purpureocillium lilacinum.

82. The autolytic yeast of claim 79, wherein the cellulolytic enzyme is β-1,3-glucomannase.

83. The autolytic yeast of claim 79, wherein the cellulolytic enzyme is targeted for extracellular secretion.

84. The autolytic yeast of claim 79, wherein the cellulolytic enzyme is targeted to an intracellular compartment.

85. The autolytic yeast of claim 79, wherein the yeast is Rhodosporidium toruloides.

86. An autolytic method of producing a bioproduct from an industrious yeast comprising:
genetically engineering an industrious yeast to express and/or secrete a cellulolytic enzyme, wherein the industrious yeast produces a bioproduct, and wherein the expression of the cellulolytic enzyme is under the control of an inducible promoter;
growing the yeast to produce the bioproduct;
inducing expression of the cellulolytic enzyme to autolyse the yeast; and
extracting, isolating, and/or purifying the bioproduct.

87. The method of claim 86, wherein the yeast is Rhodosporidium toruloides.

88. The method of claim 86 or 87, wherein the genetically engineering comprising inserting the MAN5C gene from Purpureocillium lilacinum.
